(19) United States
(12) Patent Application Publication
(10) Pub. No.: US 2024/0226101 A1
(43) Pub. Date: Jul. 11, 2024

(54) PYRIMIDINYLAMINOBENZENES FOR LUNG CANCER TREATMENT

(71) Applicant: Suzhou Puhe BioPharma Co., LTD., Suzhou (CN)

(72) Inventors: Maosheng Duan, Beijing (CN); Shuai Yuan, Suzhou (CN)

(21) Appl. No.: 18/558,522

(22) PCT Filed: May 2, 2022
(86) PCT No.: PCT/CN2022/090836

§ 371 Date: Nov. 1, 2023

(30) Foreign Application Priority Data
Apr. 30, 2021 (WO) PCT/CN2021/091463

(51) Int. Cl. A61K 31/506; A61P 35/00; C07D 239/42

(52) U.S. Cl. CPC A61K 31/506; A61P 35/00; C07D 239/42; C07B 2200/13

(57) ABSTRACT

Provided herein is a method of treating, preventing, or ameliorating one or more symptoms of lung cancer with a pyrimidinylaminobenzene, e.g., a compound of Formula (I).

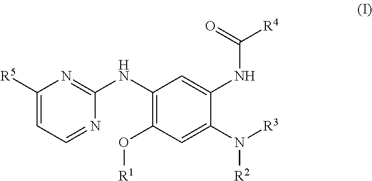

### CROSS REFERENCE TO RELATED APPLICATION

This application claims the benefit of the priority of International Application No. PCT/CN2021/091463, filed Apr. 30, 2021, under 35 U.S.C. 119(a); the disclosure of which is incorporated herein by reference in its entirety.

### FIELD

Provided herein is a method of treating, preventing, or ameliorating one or more symptoms of lung cancer with a pyrimidinylaminobenzene.

### BACKGROUND

Lung cancer is the second most commonly diagnosed cancer and the leading cause of cancer death worldwide in 2020. Sung et al., “Global cancer statistics 2020: GLOBOCAN estimates of incidence and mortality worldwide for 36 cancers in 185 countries,” CA Cancer J. Clin. 2021. Non-small cell lung cancer (NSCLC) accounts for approximately 80-85% of all lung cancers. Osmani et al., Semin. Cancer Biol. 2018, 52, 103-9. Mutations in epidermal growth factor receptor (EGFR) are common in NSCLC. Zhou et al., Hum. Genomics 2021, 15, 21; Burnett et al., PLoS One 2021, 16, e0247620. Almost 90% of all EGFR mutations are exon 19 deletion and L858R point mutations in exon 21, together known as classical EGFR mutations, and the remaining EGFR mutations include exon 20 insertion (EGFR ex20ins) (roughly 4-12%), G719X, S768I, and L861Q. Fang et al., BMC Cancer 2019, 19, 595. The EGFR ex20ins is the third most common EGFR mutation in NSCLC and associated with de novo resistance to targeted EGFR inhibitors. Wang et al., Transl. Cancer Res. 2020, 9, 2982-91. NSCLC with an EGFR ex20ins has a poor prognosis and limited treatment options. Vyse and Huang, Signal Transduct. Target Ther. 2019, 4, 5; Wang et al., Transl. Cancer Res. 2020, 9, 2982-91; Burnett et al., PLoS One 2021, 16, e0247620. Currently, clinically approved targeted EGFR inhibitors have failed to effectively treat NSCLC driven by an EGFR ex20ins. Vyse and Huang, Signal Transduct. Target Ther. 2019, 4, 5. Therefore, there is a high unmet need for an effective therapy for treating NSCLC harboring an EGFR ex20ins mutation.

### SUMMARY OF THE DISCLOSURE

Provided herein is a method of treating, preventing, or ameliorating one or more symptoms of lung cancer bearing an EGFR mutation in a subject, comprising administering to the subject in need thereof a therapeutically effective amount of a compound of Formula (I):

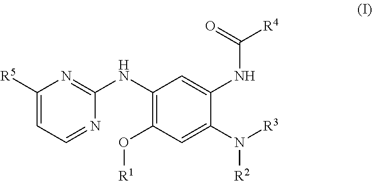

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
R¹ is C₁₋₆ alkyl or C₃₋₁₀ cycloalkyl;
R² is hydrogen or C₁₋₆ alkyl;
R³ is C₁₋₆ alkyl or heterocyclyl, each independently substituted with amino, C₁₋₆ alkylamino, di(C₁₋₆ alkyl)amino, or heterocyclyl;
R⁴ is C₂₋₆ alkenyl or C₂₋₆ alkynyl; and
R⁵ is bicyclic heteroaryl;
wherein each alkyl, alkenyl, alkynyl, cycloalkyl, heteroaryl, and heterocyclyl is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i)hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a},
wherein each Q^{a} is independently selected from: (a) deuterium, cyano, halo, nitro, imino, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —C(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, NR^{e}C(O)R^{h}, —NR^{e}C(O)OR^{f}, —NR^{e}C(O)NR^{f}R^{g}, —NR^{e}C(O)SR^{f}, NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

Additionally provided herein is a method of treating, preventing, or ameliorating one or more symptoms of lung cancer in a subject, comprising the steps of:
(a) determining the presence of an EGFR exon 20 mutation in a sample from the subject; and
(b) if the sample is determined to have an EGFR exon 20 mutation, administering to the subject a therapeutically effective amount of a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

Furthermore, provided herein is a method of inhibiting the growth of a lung cancer cell, comprising contacting the cell with an effective amount of a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

### DETAILED DESCRIPTION

To facilitate understanding of the disclosure set forth herein, a number of terms are defined below.

Generally, the nomenclature used herein and the laboratory procedures in organic chemistry, medicinal chemistry, biochemistry, biology, and pharmacology described herein are those well-known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, and mouse. The terms “subject” and “patient” are used interchangeably herein in reference, for example, to a mammalian subject, such as a human subject. In one embodiment, the subject is a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “alleviate” and “alleviating” refer to easing or reducing one or more symptoms (e.g., pain) of a disorder, disease, or condition. The terms can also refer to reducing adverse effects associated with an active ingredient. Sometimes, the beneficial effects that a subject derives from a prophylactic or therapeutic agent do not result in a cure of the disorder, disease, or condition.

The term “contacting” or “contact” is meant to refer to bringing together of a therapeutic agent and a biological molecule (e.g., a protein, enzyme, RNA, or DNA), cell, or tissue such that a physiological and/or chemical effect takes place as a result of such contact. Contacting can take place in vitro, ex vivo, or in vivo. In one embodiment, a therapeutic agent is contacted with a biological molecule in vitro to determine the effect of the therapeutic agent on the biological molecule. In another embodiment, a therapeutic agent is contacted with a cell in cell culture (in vitro) to determine the effect of the therapeutic agent on the cell. In yet another embodiment, the contacting of a therapeutic agent with a biological molecule, cell, or tissue includes the administration of a therapeutic agent to a subject having the biological molecule, cell, or tissue to be contacted.

The term “therapeutically effective amount” or “effective amount” is meant to include the amount of a compound that, when administered, is sufficient to prevent development of, or alleviate to some extent, one or more of the symptoms of the disorder, disease, or condition being treated. The term “therapeutically effective amount” or “effective amount” also refers to the amount of a compound that is sufficient to elicit a biological or medical response of a biological molecule (e.g., a protein, enzyme, RNA, or DNA), cell, tissue, system, animal, or human, which is being sought by a researcher, veterinarian, medical doctor, or clinician.

The term “IC₅₀” or “EC₅₀” refers to an amount, concentration, or dosage of a compound that is required for 50% inhibition of a maximal response in an assay that measures such a response.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of a subject (e.g., a human) without excessive toxicity, irritation, allergic response, immunogenicity, or other problems or complications, and commensurate with a reasonable benefit/risk ratio. See, e.g., Remington: The Science and Practice of Pharmacy, 23rd ed.; Adejare Ed.; Academic Press, 2020; Handbook of Pharmaceutical Excipients, 9th ed.; Sheskey et al., Eds.; Pharmaceutical Press, 2020; Handbook of Pharmaceutical Additives, 3rd ed.; Ash and Ash Eds.; Synapse Information Resources, 2007; Pharmaceutical Preformulation and Formulation, 1st ed.; Gibson Ed.; CRC Press, 2015.

The term “about” or “approxiamtely” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 25%, 20%, 15%, 10%, 9%, 8%, 7%, 6%, 5%, 4%, 3%, 2%, 1%, 0.5%, or 0.05% of a given value or range.

The term “alkyl” refers to a linear or branched saturated monovalent hydrocarbon radical, wherein the alkyl is optionally substituted with one or more substituents Q as described herein. For example, C₁₋₆ alkyl refers to a linear saturated monovalent hydrocarbon radical of 1 to 6 carbon atoms or a branched saturated monovalent hydrocarbon radical of 3 to 6 carbon atoms. In certain embodiments, the alkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms, e.g., n-propyl and isopropyl), butyl (including all isomeric forms, e.g., n-butyl, isobutyl, sec-butyl, and t-butyl), pentyl (including all isomeric forms, e.g., n-pentyl, isopentyl, sec-pentyl, neopentyl, and tert-pentyl), and hexyl (including all isomeric forms, e.g., n-hexyl, isohexyl, and sec-hexyl).

The term “alkenyl” refers to a linear or branched monovalent hydrocarbon radical, which contains one or more, in one embodiment, one, two, three, or four, in another embodiment, one, carbon-carbon double bond(s). The alkenyl is optionally substituted with one or more substituents Q as described herein. The term “alkenyl” embraces radicals having a “cis” or “trans” configuration or a mixture thereof, or alternatively, a “Z” or “E” configuration or a mixture thereof, as appreciated by those of ordinary skill in the art. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenyl groups include, but are not limited to, ethenyl, propenyl (including all isomeric forms, e.g., propen-1-yl, propen-2-yl, and allyl), and butenyl (including all isomeric forms, e.g., buten-1-yl, buten-2-yl, buten-3-yl, and 2-buten-1-yl).

The term “alkynyl” refers to a linear or branched monovalent hydrocarbon radical, which contains one or more, in one embodiment, one, two, three, or four, in another embodiment, one, carbon-carbon triple bond(s). The alkynyl is optionally substituted with one or more substituents Q as described herein. For example, C₂₋₆ alkynyl refers to a linear unsaturated monovalent hydrocarbon radical of 2 to 6 carbon atoms or a branched unsaturated monovalent hydrocarbon radical of 4 to 6 carbon atoms. In certain embodiments, the alkynyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 4 to 20 (C₄₋₂₀), 4 to 15 (C₄₋₁₅), 4 to 10 (C₄₋₁₀), or 4 to 6 (C₄₋₆) carbon atoms. Examples of alkynyl groups include, but are not limited to, ethynyl (—C≡CH), propynyl (including all isomeric forms, e.g., 1-propynyl (—C≡CCH₃) and propargyl (—CH₂C≡CH)), butynyl (including all isomeric forms, e.g., 1-butyn-1-yl and 2-butyn-1-yl), pentynyl (including all isomeric forms, e.g., 1-pentyn-1-yl and 1-methyl-2-butyn-1-yl), and hexynyl (including all isomeric forms, e.g., 1-hexyn-1-yl and 2-hexyn-1-yl).

The term “cycloalkyl” refers to a cyclic monovalent hydrocarbon radical, which is optionally substituted with one or more substituents Q as described herein. In one embodiment, the cycloalkyl is a saturated or unsaturated but non-aromatic, and/or bridged or non-bridged, and/or fused bicyclic group. In certain embodiments, the cycloalkyl has from 3 to 20 (C₃₋₂₀), from 3 to 15 (C₃₋₁₅), from 3 to 10 (C₃₋₁₀), or from 3 to 7 (C₃₋₇) carbon atoms. In one embodiment, the cycloalkyl is monocyclic. In another embodiment, the cycloalkyl is bicyclic. In yet another embodiment, the cycloalkyl is tricyclic. In still another embodiment, the cycloalkyl is polycyclic. Examples of cycloalkyl groups include, but are not limited to, cyclopropyl, cyclobutyl, cyclopentyl, cyclopentenyl, cyclohexyl, cyclohexenyl, cyclohexadienyl, cycloheptyl, cycloheptenyl, bicyclo[1.1.1]pentyl, bicyclo[2.1.1]hexyl, bicyclo[2.2.1]heptyl, bicyclo[2.2.2]octyl, decalinyl, and adamantyl.

The term “aryl” refers to a monovalent monocyclic aromatic hydrocarbon radical and/or monovalent polycyclic aromatic hydrocarbon radical that contain at least one aromatic carbon ring. In certain embodiments, the aryl has from 6 to 20 (C₆₋₂₀), from 6 to 15 (C₆₋₁₅), or from 6 to 10 (C₆₋₁₀) ring carbon atoms. Examples of aryl groups include, but are not limited to, phenyl, naphthyl, fluorenyl, azulenyl, anthryl, phenanthryl, pyrenyl, biphenyl, and terphenyl. The aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In one embodiment, the aryl is monocyclic. In another embodiment, the aryl is bicyclic. In yet another embodiment, the aryl is tricyclic. In still another embodiment, the aryl is polycyclic. In certain embodiments, the aryl is optionally substituted with one or more substituents Q as described herein.

The term “aralkyl” or “arylalkyl” refers to a monovalent alkyl group substituted with one or more aryl groups. In certain embodiments, the aralkyl has from 7 to 30 (C₇₋₃₀), from 7 to 20 (C₇₋₂₀), or from 7 to 16 (C₇₋₁₆) carbon atoms. Examples of aralkyl groups include, but are not limited to, benzyl, phenylethyl (including all isomeric forms, e.g., 1-phenylethyl and 2-phenylethyl), and phenylpropyl (including all isomeric forms, e.g., 1-phenylpropyl, 2-phenyl-propyl, and 3-phenylpropyl). In certain embodiments, the aralkyl is optionally substituted with one or more substituents Q as described herein.

The term “heteroaryl” refers to a monovalent monocyclic aromatic group or monovalent polycyclic aromatic group that contain at least one aromatic ring, wherein at least one aromatic ring contains one or more heteroatoms, each independently selected from O, S, and N, in the ring. The heteroaryl is bonded to the rest of a molecule through the aromatic ring. Each ring of a heteroaryl group can contain one or two O atoms, one or two S atoms, and/or one to four N atoms; provided that the total number of heteroatoms in each ring is four or less and each ring contains at least one carbon atom. In certain embodiments, the heteroaryl has from 5 to 20, from 5 to 15, or from 5 to 10 ring atoms. In one embodiment, the heteroaryl is monocyclic. Examples of monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. In another embodiment, the heteroaryl is bicyclic. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyrindyl (including all isomeric forms, e.g., furo[2,3-b]pyridinyl, furo[2,3-c]pyridinyl, furo[3,2-b]-pyridinyl, furo[3,2-c]pyridinyl, furo[3,4-b]pyridinyl, and furo[3,4-c]pyridinyl), imidazopyridinyl (including all isomeric forms, e.g., imidazo[1,2-a]pyridinyl, imidazo[4,5-b]pyridinyl, and imidazo[4,5-c]pyridinyl), imidazothiazolyl (including all isomeric forms, e.g., imidazo[2,1-b]-thiazolyl and imidazo[4,5-d]thiazolyl), indazolyl, indolizinyl, indolyl, isobenzofuranyl, isobenzothienyl (i.e., benzo[c]thienyl), isoindolyl, isoquinolinyl, naphthyridinyl (including all isomeric forms, e.g., 1,5-naphthyridinyl, 1,6-naphthyridinyl, 1,7-naphthyridinyl, and 1,8-naphthyridinyl), oxazolopyridinyl (including all isomeric forms, e.g., oxazolo[4,5-b]pyridinyl, oxazolo[4,5-c]-pyridinyl, oxazolo[5,4-b]pyridinyl, and oxazolo[5,4-c]pyridinyl), phthalazinyl, pteridinyl, purinyl, pyrrolopyridyl (including all isomeric forms, e.g., pyrrolo[2,3-b]pyridinyl, pyrrolo[2,3-c]pyridinyl, pyrrolo[3,2-b]pyridinyl, and pyrrolo[3,2-c]pyridinyl), quinolinyl, quinoxalinyl, quinazolinyl, thiadiazolopyrimidyl (including all isomeric forms, e.g., [1,2,5]thiadiazolo[3,4-d]-pyrimidinyl and [1,2,3]thiadiazolo[4,5-d]pyrimidinyl), and thienopyridyl (including all isomeric forms, e.g., thieno[2,3-b]pyridinyl, thieno[2,3-c]pyridinyl, thieno[3,2-b]pyridinyl, and thieno-[3,2-c]pyridinyl). In yet another embodiment, the heteroaryl is tricyclic. Examples of tricyclic heteroaryl groups include, but are not limited to, acridinyl, benzindolyl, carbazolyl, dibenzo-furanyl, perimidinyl, phenanthrolinyl, phenanthridinyl (including all isomeric forms, e.g., 1,5-phenanthrolinyl, 1,6-phenanthrolinyl, 1,7-phenanthrolinyl, 1,9-phenanthrolinyl, and 2,10-phenanthrolinyl), phenarsazinyl, phenazinyl, phenothiazinyl, phenoxazinyl, and xanthenyl. In certain embodiments, the heteroaryl is optionally substituted with one or more substituents Q as described herein.

The term “heterocyclyl” or “heterocyclic” refers to a monovalent monocyclic non-aromatic ring system or monovalent polycyclic ring system that contains at least one non-aromatic ring, wherein one or more of the non-aromatic ring atoms are heteroatoms, each independently selected from O, S, and N; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. The heterocyclyl is bonded to the rest of a molecule through the non-aromatic ring. In certain embodiments, the heterocyclyl is a monocyclic, bicyclic, tricyclic, or tetracyclic ring system, which may be fused or bridged, and in which nitrogen or sulfur atoms may be optionally oxidized, nitrogen atoms may be optionally quaternized, and some rings may be partially or fully saturated, or aromatic. The heterocyclyl may be attached to the main structure at any heteroatom or carbon atom which results in the creation of a stable compound. Examples of heterocyclyls and heterocyclic groups include, but are not limited to, azepinyl, benzodioxanyl, benzodioxolyl, benzofuranonyl, chromanyl, decahydroisoquinolinyl, dihydrobenzofuranyl, dihydrobenzisothiazolyl, dihydrobenzisoxazinyl (including all isomeric forms, e.g., 1,4-dihydrobenzo[d][1,3]oxazinyl, 3,4-dihydrobenzo[c][1,2]-oxazinyl, and 3,4-dihydrobenzo[d][1,2]oxazinyl), dihydrobenzothienyl, dihydroisobenzofuranyl, dihydrobenzo[c]thienyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isochromanyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, thiochromanyl, tetrahydroquinolinyl, and 1,3,5-trithianyl. In certain embodiments, the heterocyclyl is optionally substituted with one or more substituents Q as described herein.

The term “halogen”, “halide,” or “halo” refers to fluoro, chloro, bromo, and/or iodo.

The term “optionally substituted” is intended to mean that a group or substituent, such as an alkyl, alkenyl, alkynyl, cycloalkyl, aryl, aralkyl, heteroaryl, or heterocyclyl group, may be substituted with one or more, in one embodiment, one, two, three, or four, substituents Q, each of which is independently selected from, e.g., (a) deuterium (-D), cyano (—CN), halo, imino (═NH), nitro (—NO₂), and oxo (═O); (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —C(NR^{a})NR^{b}R^{c}, —OC(S)R^{a}, —OC(S)OR^{a}, —OC(S)NR^{b}R^{c}, —OS(O)R^{a}, —OS(O₂)^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}, or (iii) R^{b} and R^{c} together with the N atom to which they are attached form heterocyclyl optionally substituted with one or more, in one embodiment, one, two, three, or four, substituents Q^{a}. As used herein, all groups that can be substituted are “optionally substituted.”

In one embodiment, each Q^{a} is independently selected from: (a) deuterium, cyano, halo, imino, nitro, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —C(O)R^{e}, —C(O)OR^{e}, —OC(O)NR^{f}R^{g}, —OC(O)SR^{e}, —OC(NR^{e})NR^{f}R^{g}, —OC(S)R^{e}, —OC(S)OR^{e}, —C(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, NR^{e}S(O)R^{h}, NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, “optically active” and “enantiomerically active” refer to a collection of molecules, which has an enantiomeric excess of no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, an optically active compound comprises about 95% or more of one enantiomer and about 5% or less of the other enantiomer based on the total weight of the enantiomeric mixture in question. In certain embodiments, an optically active compound comprises about 98% or more of one enantiomer and about 2% or less of the other enantiomer based on the total weight of the enantiomeric mixture in question. In certain embodiments, an optically active compound comprises about 99% or more of one enantiomer and about 1% or less of the other enantiomer based on the total weight of the enantiomeric mixture in question.

In describing an optically active compound, the prefixes R and S are used to denote the absolute configuration of the compound about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the compound, R and S.

The term “isotopically enriched” refers to a compound that contains an unnatural proportion of an isotope at one or more of the atoms that constitute such a compound. In certain embodiments, an isotopically enriched compound contains unnatural proportions of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹³O), oxygen-16 (¹¹O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an isotopically enriched compound is in a stable form, that is, non-radioactive. In certain embodiments, an isotopically enriched compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an isotopically enriched compound is in an unstable form, that is, radioactive. In certain embodiments, an isotopically enriched compound contains unnatural proportions of one or more isotopes, including, but not limited to, tritium (³H), carbon-1 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²⁵I), iodine-129 (¹²⁹I), and iodine-131 (¹³¹I). It will be understood that, in a compound as provided herein, any hydrogen can be 21H as example, or any carbon can be ¹³C, as example, or any nitrogen can be ¹⁵N, as example, or any oxygen can be ¹⁸O, as example, where feasible according to the judgment of one of ordinary skill in the art.

The term “isotopic enrichment” refers to the percentage of incorporation of a less prevalent isotope (e.g., D for deuterium or hydrogen-2) of an element at a given position in a molecule in the place of a more prevalent isotope (e.g., ¹H for protium or hydrogen-1) of the element. As used herein, when an atom at a particular position in a molecule is designated as a particular less prevalent isotope, it is understood that the abundance of that isotope at that position is substantially greater than its natural abundance.

The term “isotopic enrichment factor” refers the ratio between the isotopic abundance in an isotopically enriched compound and the natural abundance of a specific isotope.

The term “hydrogen” or the symbol “H” refers to the composition of naturally occurring hydrogen isotopes, which include protium (¹H), deuterium (²H or D), and tritium (³H), in their natural abundances. Protium is the most common hydrogen isotope having a natural abundance of more than 99.98%. Deuterium is a less prevalent hydrogen isotope having a natural abundance of about 0.0156%.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156% on average, deuterium enrichment at any position in a compound synthesized using non-enriched starting materials is about 0.0156% on average. As used herein, when a particular position in an isotopically enriched compound is designated as having deuterium, it is understood that the abundance of deuterium at that position in the compound is substantially greater than its natural abundance (0.0156%).

The term “carbon” or the symbol “C” refers to the composition of naturally occurring carbon isotopes, which include carbon-12 (¹²C) and carbon-13 (¹³C) in their natural abundances. Carbon-12 is the most common carbon isotope having a natural abundance of more than 98.89%. Carbon-13 is a less prevalent carbon isotope having a natural abundance of about 1.11%.

The term “carbon-13 enrichment” or “¹³C enrichment” refers to the percentage of incorporation of carbon-13 at a given position in a molecule in the place of carbon. For example, carbon-13 enrichment of 10% at a given position means that 10% of molecules in a given sample contain carbon-13 at the specified position. Because the naturally occurring distribution of carbon-13 is about 1.11% on average, carbon-13 enrichment at any position in a compound synthesized using non-enriched starting materials is about 1.11% on average. As used herein, when a particular position in an isotopically enriched compound is designated as having carbon-13, it is understood that the abundance of carbon-13 at that position in the compound is substantially greater than its natural abundance (1.11%).

The terms “substantially pure” and “substantially homogeneous” mean, when referred to a substance, sufficiently homogeneous to appear free of readily detectable impurities as determined by a standard analytical method used by one of ordinary skill in the art, including, but not limited to, thin layer chromatography TC), gel electrophoresis, high performance liquid chromatography (HPLC), gas chromatography (GC), nuclear magnetic resonance (NMR), and mass spectrometry (MS); or sufficiently pure such that further purification would not detectably alter the physical, chemical, biological, and/or pharmacological properties, such as enzymatic and biological activities, of the substance. In certain embodiments, “substantially pure” or “substantially homogeneous” refers to a collection of molecules, wherein at least about 95%, at least about 96%, at least about 97%, at least about 98%, at least about 99%, or at least about 99.5% by weight of the molecules are a single compound, including a single enantiomer, a racemic mixture, or a mixture of enantiomers, as determined by standard analytical methods. As used herein, when an atom at a particular position in an isotopically enriched molecule is designated as a particular less prevalent isotope, a molecule that contains other than the designated isotope at the specified position is an impurity with respect to the isotopically enriched compound. Thus, for a deuterated compound that has an atom at a particular position designated as deuterium, a compound that contains a protium at the same position is an impurity.

The term “solvate” refers to a complex or aggregate formed by one or more molecules of a solute, e.g., a compound provided herein, and one or more molecules of a solvent, which are present in stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

The phrase “an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof” has the same meaning as the phrase “(i) an enantiomer, a mixture of enantiomers, a diastereomer, a mixture of two or more diastereomers, a tautomer, a mixture of two or more tautomers, or an isotopic variant of the compound referenced therein; or (ii) a pharmaceutically acceptable salt, solvate, hydrate, or prodrug of the compound referenced therein, or (iii) a pharmaceutically acceptable salt, solvate, hydrate, or prodrug of an enantiomer, a mixture of enantiomers, a diastereomer, a mixture of two or more diastereomers, a tautomer, a mixture of two or more tautomers, or an isotopic variant of the compound referenced therein.”

### Compounds

In one embodiment, described herein is a compound of Formula (I):

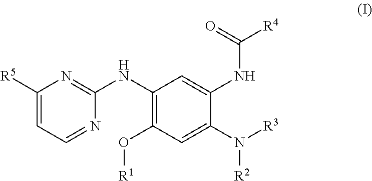

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
R¹ is C₁₋₆ alkyl or C₃₋₁₀ cycloalkyl;
R² is hydrogen or C₁₋₆ alkyl;
R³ is C₁₋₆ alkyl or heterocyclyl, each independently substituted with amino, C₁₋₆ alkylamino, di(C₁₋₆ alkyl)amino, or heterocyclyl;
R⁴ is C₂₋₆ alkenyl or C₂₋₆ alkynyl; and
R⁵ is bicyclic heteroaryl;
wherein each alkyl, alkenyl, alkynyl, cycloalkyl, heteroaryl, and heterocyclyl is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₂₋₆ alkenyl. C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆alkyl, C₂₋₄ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, in Formula (I), R⁵ is 5,6- or 6,6-fused heteroaryl, each optionally substituted with one or more substituents Q. In certain embodiments, in Formula (I), R⁵ is 5,6-fused heteroaryl, optionally substituted with one or more substituents Q. In certain embodiments, in Formula (I), R⁵ is

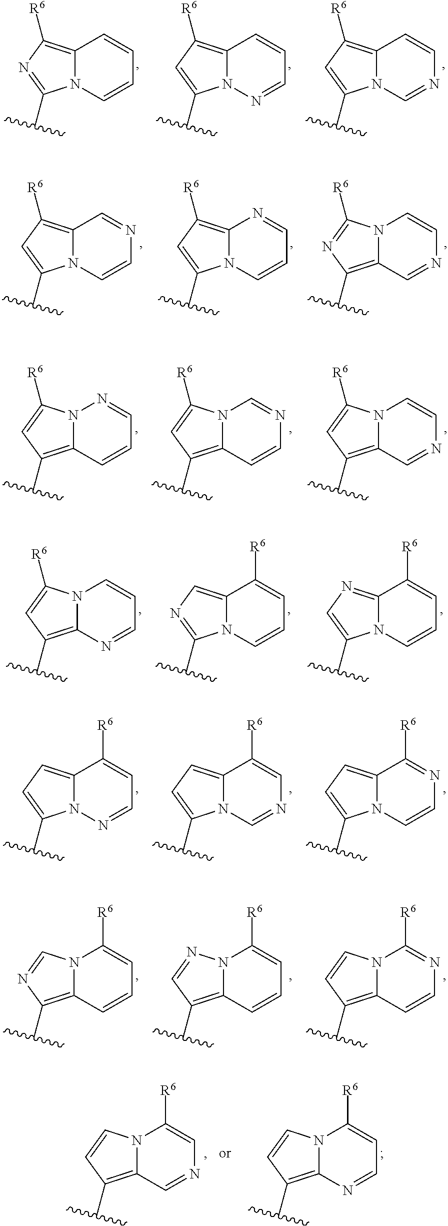

wherein each R⁶ is independently (i) hydrogen or halo; or (ii) C₁₋₆ alkyl or C₁₋₆ alkoxy, each optionally substituted with one or more substituents Q.

In another embodiment, described herein is a compound of Formula (II):

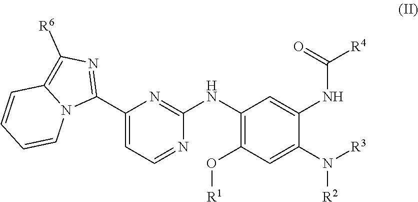

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof: wherein R¹, R², R³, R⁴, and R⁶ am each as defined herein.

In yet another embodiment, described herein is a compound of Formula (III):

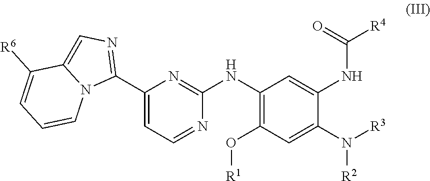

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R². R³, R⁴, and R⁶ are each as defined herein.

In yet another embodiment, described herein is a compound of Formula (IV):

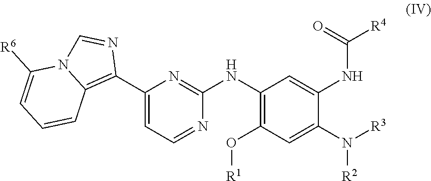

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R². R³, R⁴, and R⁶ are each as defined herein.

In yet another embodiment, described herein is a compound of Formula (V):

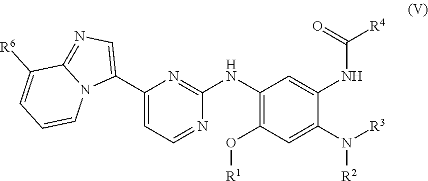

or an enantiomer, a mixture of enantiomers, a diastereomer, a mixture of two or more diasteromers, a tautomer, a mixture of two or more tautomers, or an isotopic variant thereof: or a pharmaceutically acceptable salt, solvate, hydrate, or prodrug thereof; wherein R¹, R², R³, R⁴, and R⁶ are each as defined herein.

In yet another embodiment, described herein is a compound of Formula (VI):

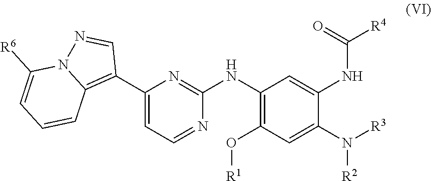

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof: wherein R¹, R². R³, R⁴, and R⁶ are each as defined herein.

In still another embodiment, described herein is a compound of Formula (VII):

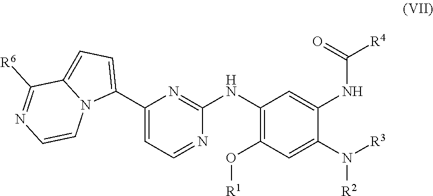

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R⁴, and R⁶ are each as defined herein.

In certain embodiments, in any one of Formulae (I) to (VII), R¹ is C₁₋₆ alkyl, optionally substituted with one or more substituents Q. In certain embodiments, in any one of Formulae (I) to (VII), R¹ is C₁₋₆ alkyl, optionally substituted with one or more halo. In certain embodiments, in any one of Formulae (I) to (VII), R¹ is C₃₋₁₀ cycloalkyl, optionally substituted with one or more substituents Q. In certain embodiments, in any one of Formulae (I) to (VII). R¹ is methyl, fluoromethyl, difluoromethyl, trifluoromethyl, ethyl, or cyclopropyl.

In certain embodiments, in any one of Formulae (I) to (VII), R² is hydrogen. In certain embodiments, in any one of Formulae (I) to (VII), R² is C₁₋₆ alkyl, optionally substituted with one or more substituents Q. In certain embodiments, in any one of Formulae (I) to (VII), R² is methyl.

In certain embodiments, in any one of Formulae (I) to (VII), R³ is C₁₋₆ alkyl, substituted with amino, C₁₋₆ alkylamino, di(C₁₋₆ alkyl)amino, or heterocyclyl; wherein each alkyl and heterocyclyl is optionally substituted with one or more substituents Q. In certain embodiments, in any one of Formulae (I) to (VII), R³ is C₁₋₆ alkyl substituted with amino. In certain embodiments, in any one of Formulae (I) to (VII), R³ is C₁₋₆ alkyl substituted with C₁₋₆ alkylamino, which is optionally substituted with one or more substituents Q. In certain embodiments, in any one of Formulae (I) to (VII), R³ is C₁₋₆ alkyl substituted with di(C₁₋₆ alkyl)amino, which is optionally substituted with one or more substituents Q. In certain embodiments, in any one of Formulae (I) to (VII), R³ is C₁₋₆ alkyl substituted with heterocyclyl, which is optionally substituted with one or more substituents Q. In certain embodiments, in any one of Formulae (I) to (VI), R³ is C₁₋₆ alkyl, substituted with methylamino or dimethylamino. In certain embodiments, in any one of Formulae (I) to (VII), R³ is 2-dimethylaminoethyl or 2-morpholin-4-ylethyl.

In certain embodiments, in any one of Formulae (I) to (VII), R³ is heterocyclyl, substituted with amino, C₁₋₆ alkylamino, or di(C₁₋₆ alkyl)amino; wherein each alkyl is optionally substituted with one or more substituents Q. In certain embodiments, in any one of Formulae (I) to (VII), R³ is 3- to 8-membered heterocyclyl, each substituted with amino, C₁₋₆ alkylamino, or di(C₁₋₆ alkyl)amino; wherein each alkyl is optionally substituted with one or more substituents Q. In certain embodiments, in any one of Formulae (I) to (VII), R³ is heterocyclyl substituted with methylamino or dimethylamino. In certain embodiments, in any one of Formulae (I) to (VII), R³ is 4-, 5-, or 6-membered heterocyclyl, each independently substituted with methylamino or dimethylamino. In certain embodiments, in any one of Formulae (I) to (VII), R³ is heterocyclyl substituted with dimethylamino. In certain embodiments, in any one of Formulae (I) to (VII), R³ is 4-, 5-, or 6-membered heterocyclyl, each independently substituted with dimethylamino. In certain embodiments, in any one of Formulae (I) to (VII), R³ is dimethylaminoazetidinyl, dimethylaminopyrrolidinyl, dimethylaminopiperidinyl, or methylpiperidinyl. In certain embodiments, in any one of Formulae (I) to (VII), R³ is 3-dimethylaminoazetidin-1-yl, 3-dimethylaminopyrrolidin-1-yl, 4-dimethylaminopiperidin-1-yl, or 1-methylpiperidin-3-yl.

In certain embodiments, in any one of Formulae (I) to (VII), R⁴ is C₂ alkenyl, optionally substituted with one or more substituents Q. In certain embodiments, in any one of Formulae (I) to (VII), R⁴ is C₂₋₆ alkenyl, optionally substituted with dimethylamino. In certain embodiments, in any one of Formulae (I) to (VII), R⁴ is ethenyl or propenyl, each optionally substituted with dimethylamino. In certain embodiments, in any one of Formulae (I) to (VII), R⁴ is ethenyl or 1-propenyl, each optionally substituted with dimethylamino. In certain embodiments, in any one of Formulae (I) to (VII), R⁴ is ethenyl or (3-dimethylamino)propen-1-yl.

In certain embodiments, in any one of Formulae (I) to (VII), R⁴ is C₂₋₆ alkynyl, optionally substituted with one or more substituents Q. In certain embodiments, in any one of Formulae (I) to (VII), R⁴ is C₂₋₆ alkynyl, optionally substituted with dimethylamino. In certain embodiments, in any one of Formulae (I) to (VII), R⁴ is ethynyl or propynyl, each optionally substituted with dimethylamino. In certain embodiments, in any one of Formulae (I) to (VII), R⁴ is ethynyl, propyn-1-yl, or (3-dimethylamino)propyn-1-yl.

In certain embodiments, in any one of Formulae (II) to (VII), R⁶ is hydrogen. In certain embodiments, in any one of Formulae (II) to (VII), R⁶ is halo. In certain embodiments, in any one of Formulae (II) to (VII). R⁶ is fluor or chloro. In certain embodiments, in any one of Formulae (II) to (VII), R⁶ is C₁₋₆ alkyl, optionally substituted with one or more substituents Q. In certain embodiments, in any one of Formulae (II) to (VII), R⁶ is methyl. In certain embodiments, in any one of Formulae (II) to (VII), R⁶ is C₁₋₆ alkoxy, optionally substituted with one or more substituents Q. In certain embodiments, in any one of Formulae (II) to (VII), R⁶ is methoxy. In certain embodiments, in any one of Formulae (II) to (VI), R⁶ is chloro, methyl, or methoxy.

In one embodiment, described herein is:

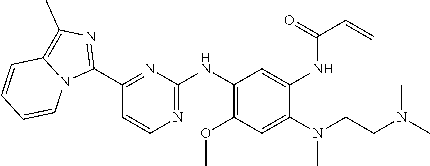 [SMILES: C=CC(=O)Nc1cc(Nc2nccc(-c3nc(C)c4ccccn34)n2)c(OC)cc1N(C)CCN(C)C]

N-(2-((2-(dimethylamino)ethyl)(methyl)amino)-4-methoxy-5-((4-(1-methylimidazo[1,5-a]-pyridin-3-yl)pyrimidin-2-yl)amino) n y)acrylamide A1;

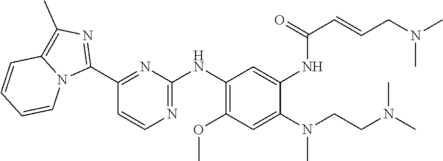 [SMILES: COc1cc(N(C)CCN(C)C)c(NC(=O)/C=C/CN(C)C)cc1Nc1nccc(-c2nc(C)c3ccccn23)n1]

(E)-4-(dimethylamino)-N-(2-((2-(dimethylamino)ethyl)(methyl)amino)-4-methoxy-5-((4-(1-methylimidazol[1,5-a]pyridin-3-yl)pyrimidin-2-yl)amino)phenyl)but-2-enamide A2;

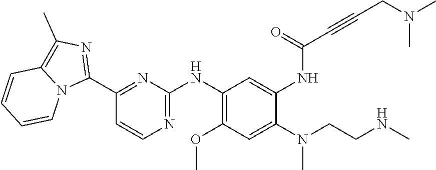 [SMILES: CNCCN(C)c1cc(OC)c(Nc2nccc(-c3nc(C)c4ccccn34)n2)cc1NC(=O)C#CCN(C)C]

4-(dimethylamino)-N-(4-methoxy-2-(methyl(2-(methylamino)ethyl)amino)-5-((4-(1-methyl-imidazo([1,5-a]pyridin-3-yl)pyrimidin-2-yl)amino)phenyl)but-2-ynamide A3;

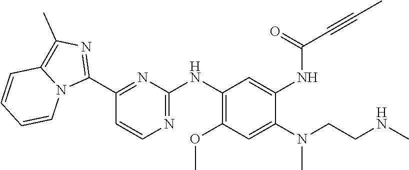 [SMILES: CC#CC(=O)Nc1cc(Nc2nccc(-c3nc(C)c4ccccn34)n2)c(OC)cc1N(C)CCNC]

N-(4-methoxy-2-(methyl(2-(methylamino)ethyl)amino)-5-((4-(1-methylimidazo[1,5-a]-pyridin-3-yl)pyrimidin-2-yl)amino)phenyl)but-2-ynamide A4;

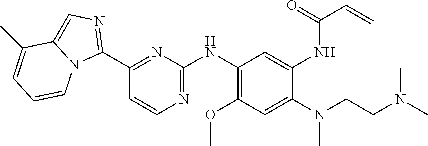

N-(2-((2-(dimethylamino)ethyl)(methyl)amino)-4-methoxy-5-((4-(8-methylimidazo[1,5-a]-pyridin-3-yl)pyrimidin-2-yl)amino)phenyl)acrylamide A5;

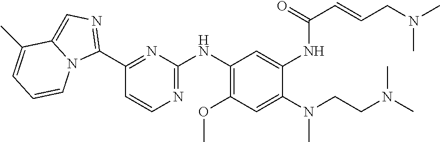

(E)-4-(dimethylamino)-N-(2-((2-(dimethylamino)ethyl)(methyl)amino)-4-methoxy-5-((4-(8-methylimidazo[1,5-a]pyridin-3-yl)pyrimidin-2-yl)amino)phenyl)but-2-enamide A6;

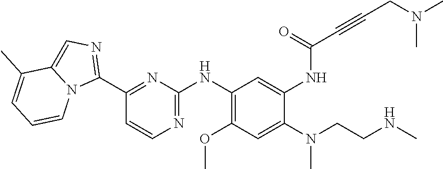

4-(dimethylamino)-N-(4-methoxy-2-(methyl(2-(methylamino)ethyl)amino)-5-((4-(8-methyl-imidazo[1,5-a]pyridin-3-yl)pyrimidin-2-yl)amino)phenyl)but-2-ynamide A7;

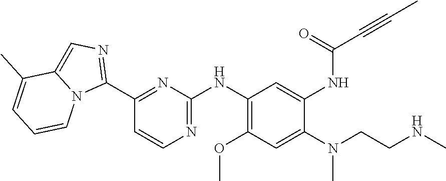

N-(4-methoxy-2-(methyl(2-(methylamino)ethyl)amino)-5-((4-(8-methylimidazo[1,5-a]-pyridin-3-yl)pyrimidin-2-yl)amino)phenyl)but-2-ynamide A8;

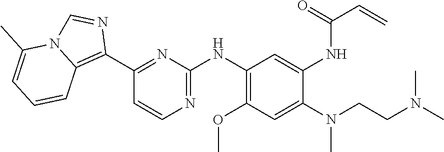

N-(2-((2-(dimethylamino)ethyl)(methyl)amino)-4-methoxy-5-((4-(5-methylimidazo[1,5-a]-pyridin-1-yl)pyrimidin-2-yl)amino)phenyl)acrylamide A9;

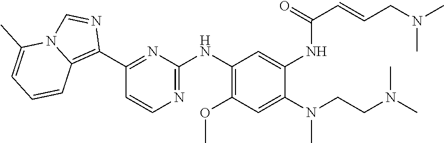

(E)-4-(dimethylamino)-N-(2-((2-(dimethylamino)ethyl)(methyl)amino)-4-methoxy-5-((4-(5-methylimidazo[1,5-a]pyridin-1-yl)pyrimidin-2-yl)amino)phenyl)but-2-enamide A10;

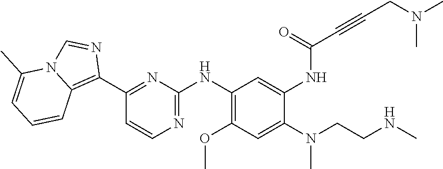

4-(dimethylamino)-N-(4-methoxy-2-(methyl(2-(methylamino)ethyl)amino)-5-((4-(5-methyl-imidazo[1,5-a]pyridin-1-yl)pyrimidin-2-yl)amino)phenyl)but-2-ynamide A11;

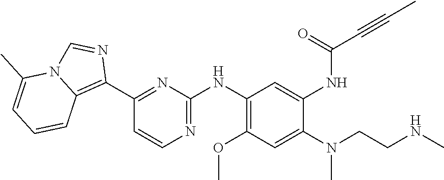

N-(4-methoxy-2-(methyl(2-(methylamino)ethyl)amino)-5-((4-(5-methylimidazo[1,5-a]-pyridin-1-yl)pyrimidin-2-yl)amino)phenyl)but-2-ynamide A12;

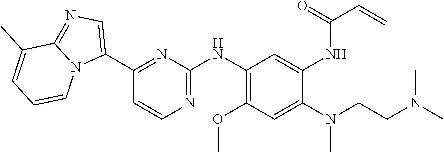

N-(2-((2-(dimethylamino)ethyl)(methyl)amino)-4-methoxy-5-((4-(8-methylimidazo[1,2-a]-pyridin-3-yl)pyrimidin-2-yl)amino)phenyl)acrylamide A13;

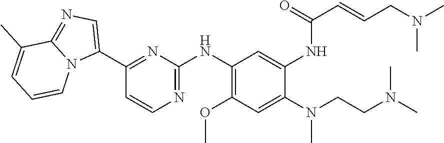

(E)-4-(dimethylamino)-N-(2-((2-(dimethylamino)ethyl)(methyl)amino)-4-methoxy-5-((4-(8-methylimidazo[1,2-a]pyridin-3-yl)pyrimidin-2-yl)amino)phenyl)but-2-enamide A14;

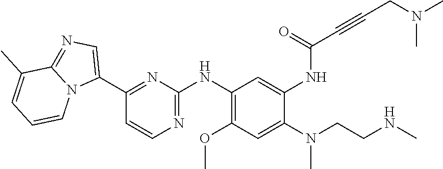

4-(dimethylamino)-N-(4-methoxy-2-(methyl(2-(methylamino)ethyl)amino)-5-((4-(8-methylimidazo[1,2-a]pyridin-3-yl)pyrimidin-2-yl)amino)phenyl)but-2-ynamide A15;

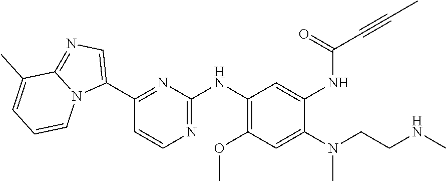

N-(4-methoxy-2-(methyl(2-(methylamino)ethyl)amino)-5-((4-(8-methylimidazo[1,2-a]-pyridin-3-yl)pyrimidin-2-yl)amino)phenyl)but-2-ynamide A16;

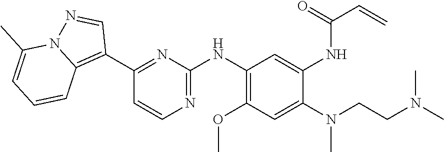

N-(2-((2-(dimethylamino)ethyl)(methyl)amino)-4-methoxy-5-((4-(7-methylpyrazolo[1,5-a]-pyridin-3-yl)pyrimidin-2-yl)amino)phenyl)acrylamide A17;

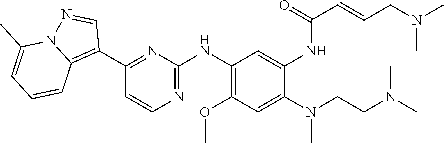

(E)-4-(dimethylamino)-N-(2-((2-(dimethylamino)ethyl)(methyl)amino)-4-methoxy-5-((4-(7-methylpyrazolo[1,5-a]pyridin-3-yl)pyrimidin-2-yl)amino)phenyl)but-2-enamide A18;

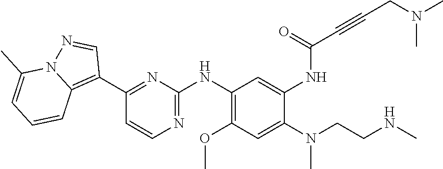

4-(dimethylamino)-N-(4-methoxy-2-(methyl(2-(methylamino)ethyl)amino)-5-((4-(7-methylpyrazolo[1,5-a]pyridin-3-yl)pyrimidin-2-yl)amino)phenyl)but-2-ynamide A19;

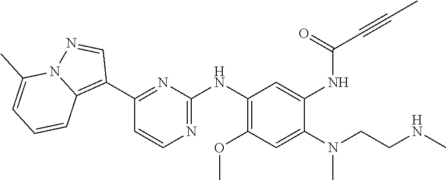

N-(4-methoxy-2-(methyl(2-(methylamino)ethyl)amino)-5-((4-(7-methylpyrazolo[1,5-a]-pyridin-3-yl)pyrimidin-2-yl)amino)phenylbut-2-ynamide A20;

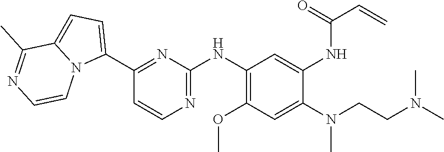

N-(2-((2-(dimethylamino)ethyl)(methyl)amino)-4-methoxy-5-((4-(1-methylpyrrolo[1,2-a]-pyrazin-6-yl)pyrimidin-2-yl)amino)phenyl)acrylamide A21;

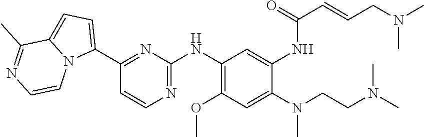

(E)-4-(dimethylamino)-N-(2-((2-(dimethylamino)ethyl)(methyl)amino)-4-methoxy-5-((4-(1-methylpyrrolo[1,2-a]pyrazin-6-yl)pyrimidin-2-yl)amino)phenyl)but-2-enamide A22;

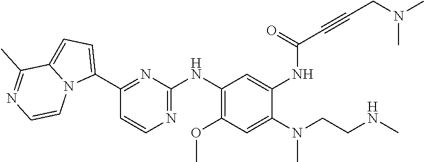

4-(dimethylamino)-N-(4-methoxy-2-(methyl(2-(methylamino)ethyl)amino)-5-((4-(1-methyl-pyrrolo[1,2-a]pyrazin-6-yl)pyrimidin-2-yl)amino)phenyl)but-2-ynamide A23;

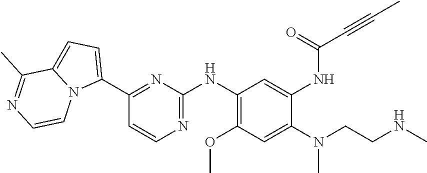

N-(4-methoxy-2-(methyl(2-(methylamino)ethyl)amino)-5-((4-(I-methylpyrrolo[1,2-a]-pyrazin-6-yl)pyrimidin-2-yl)amino)phenyl)but-2-ynamide A24;

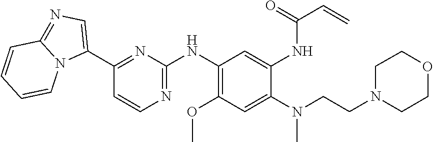 [SMILES: C=CC(=O)Nc1cc(Nc2nccc(-c3cnc4ccccn34)n2)c(OC)cc1N(C)CCN1CCOCC1]

N-(5-((4-(imidazo[1,2-a]pyridin-3-yl)pyrimidin-2-yl)amino)-4-methoxy-2-(methyl(2-morpholinoethyl)amino)phenyl)acrylamide A25;

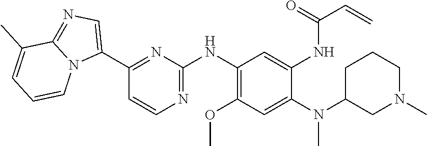

N-(4-methoxy-2-(methyl(1-methylpiperidin-3-yl)amino)-5-((4-(8-methylimidazo[1,2-a]-pyridin-3-yl)pyrimidin-2-yl)amino)phenyl)acrylamide A26;

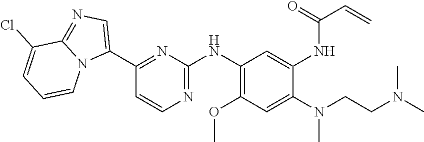 [SMILES: C=CC(=O)Nc1cc(Nc2nccc(-c3cnc4c(Cl)cccn34)n2)c(OC)cc1N(C)CCN(C)C]

N-(5-((4-(8-chloroimidazo[1,2-a]pyridin-3-yl)pyrimidin-2-yl)amino)-2-((2-(dimethylamino)-ethyl)(methyl)amino)-4-methoxyphenyl)acrylamide A27;

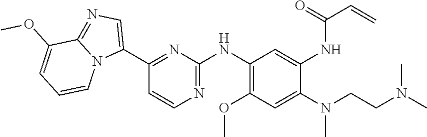

N-(2-((2-(dimethylamino)ethyl)(methyl)amino)-4-methoxy-5-((4-(8-methoxyimidazo[1,2-a]-pyridin-3-yl)pyrimidin-2-yl)amino)phenyl)acrylamide A28; or

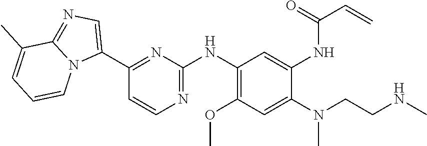

N-(4-methoxy-2-(methyl(2-(methylamino)ethyl)amino)-5-((4-(8-methylimidazo[1,2-a]-pyridin-3-yl)pyrimidin-2-yl)amino)phenyl)acrylamide A29;
   or a tautomer, a mixture of two or more tautomers, or an isotopic variant thereof; or a pharmaceutically acceptable salt, solvate, hydrate, or prodrug thereof.

In another embodiment, described herein is N-(2-((2-(dimethylamino)ethyl)-(methyl)amino)-4-methoxy-5-((4-(8-methylimidazo[1,2-a]pyridin-3-yl)pyrimidin-2-yl)amino)phenyl)acrylamide A13 (“compound A13”); or a tautomer, a mixture of two or more tautomers, or an isotopic variant thereof; or a pharmaceutically acceptable salt, solvate, hydrate, or prodrug thereof. In certain embodiments, described herein is compound A13, or a pharmaceutically acceptable solvate or hydrate thereof. In certain embodiments, compound A13 is in a crystalline form. In certain embodiments, compound A13 is in a crystalline form having an X-ray powder diffractogram comprising peaks at two-theta angles (°) of approximately 6.6, 13.8, and 19.5. In certain embodiments, compound A13 is in a crystalline form having an X-ray powder diffractogram comprising peaks at two-theta angles (°) of approximately 6.6, 10.3, 13.8, 15.2, 16.0, 16.4, 17.1, 19.5, 20.0, 21.2, 22.0, 22.7, 24.3, 25.0, 25.9, and 27.2.

In yet another embodiment, described herein is a pharmaceutically acceptable salt of compound A13; or a tautomer, a mixture of two or more tautomers, or an isotopic variant thereof; or a pharmaceutically acceptable solvate or hydrate thereof. In certain embodiments, the pharmaceutically acceptable salt of compound A13 is crystalline.

In yet another embodiment, described herein is a benzoate, fumarate, hydrochloride, maleate, mesylate, succinate, or L-tartrate salt of compound A13; or a pharmaceutically acceptable solvate or hydrate thereof.

In yet another embodiment, described herein is a benzoate salt of compound A13; or a pharmaceutically acceptable solvate or hydrate thereof. In certain embodiments, the benzoate salt of compound A13 is crystalline. In certain embodiments, the benzoate salt of compound A13 is in a crystalline form having an X-ray powder diffractogram comprising peaks at two-theta angles (°) of approximately 8.7, 10.3, 16.6, and 20.5. In certain embodiments, the benzoate salt of compound A13 is in a crystalline form having an X-ray powder diffractogram comprising peaks at two-theta angles (°) of approximately 8.0, 8.7, 10.3, 14.5, 16.6, 17.4, 18.0, 19.7, 20.5, 22.9, and 23.4.

In yet another embodiment, described herein is a fumarate salt of compound A13; or a pharmaceutically acceptable solvate or hydrate thereof. In certain embodiments, the fumarate salt is crystalline. In certain embodiments, the fumarate salt of compound A13 is in a crystalline form having an X-ray powder diffractogram comprising peaks at two-theta angles (°) of approximately 6.3, 14.8, and 16.1. In certain embodiments, the fumarate salt of compound A13 is in a crystalline form having an X-ray powder diffractogram comprising peaks at two-theta angles (°) of approximately 6.3, 6.9, 9.8, 11.6, 13.6, 14.8, 16.1, 19.0, 19.7, 20.9, 22.3, 23.7, and 26.0.

In yet another embodiment, described herein is a hydrochloride salt of compound A13; or a pharmaceutically acceptable solvate or hydrate thereof. In certain embodiments, the hydrochloride salt of compound A13 is crystalline. In certain embodiments, the hydrochloride salt is in a crystalline form having an X-ray powder diffractogram comprising peaks at two-theta angles (°) of approximately 3.6, 22.1, and 25.9. In certain embodiments, the hydrochloride salt of compound A13 is in a crystalline form having an X-ray powder diffractogram comprising peaks at two-theta angles (°) of approximately 3.6, 7.3, 10.0, 11.0, 11.3, 18.4, 22.1, 25.9, 27.1, and 29.6.

In yet another embodiment, described herein is a maleate salt of compound A13; or a pharmaceutically acceptable solvate or hydrate thereof. In certain embodiments, the maleate salt of compound A13 is crystalline. In certain embodiments, the maleate salt of compound A13 is in a crystalline form having an X-ray powder diffractogram comprising peaks at two-theta angles (°) of approximately 8.6, 10.3, and 23.3. In certain embodiments, the maleate salt of compound A13 is in a crystalline form having an X-ray powder diffractogram comprising peaks at two-theta angles (°) of approximately 5.4, 8.0, 8.6, 10.3, 13.0, 13.6, 13.8, 14.4, 14.8, 15.9, 16.7, 17.2, 17.9, 19.0, 19.9, 20.7, 23.3, 26.1, and 27.1.

In yet another embodiment, described herein is a mesylate salt of compound A13; or a pharmaceutically acceptable solvate or hydrate thereof. In certain embodiments, the mesylate salt of compound A13 is crystalline. In certain embodiments, the mesylate salt of compound A13 is in a crystalline form having an X-ray powder diffractogram comprising peaks at two-theta angles (°) of approximately 4.0, 16.2, and 19.8. In certain embodiments, the mesylate salt of compound A13 is in a crystalline form having an X-ray powder diffractogram comprising peaks at two-theta angles (°) of approximately 4.0, 10.0, 11.0, 12.1, 16.2, 18.4, 19.8, 20.3, 21.6, 22.5, 24.5, 29.6, and 33.7.

In yet another embodiment, described herein is a succinate salt of compound A13; or a pharmaceutically acceptable solvate or hydrate thereof. In certain embodiments, the succinate salt of compound A13 is crystalline. In certain embodiments, the succinate salt of compound A13 is in a crystalline form having an X-ray powder diffractogram comprising peaks at two-theta angles (°) of approximately 9.8, 22.1, and 24.6. In certain embodiments, the succinate salt of compound A13 is in a crystalline form having an X-ray powder diffractogram comprising peaks at two-theta angles (°) of approximately 6.9, 9.3, 9.8, 12.1, 12.7, 14.7, 15.6, 16.7, 17.7, 20.1, 20.8, 22.1, 23.5, 24.6, 26.0, 28.2, and 29.6.

In still another embodiment, described herein is an L-tartrate salt of compound A13; or a pharmaceutically acceptable solvate or hydrate thereof. In certain embodiments, the L-tartrate salt of compound A13 is crystalline. In certain embodiments, the L-tartrate salt of compound A13 is in a crystalline form having an X-ray powder diffractogram comprising peaks at two-theta angles (−) of approximately 8.6, 17.3, and 21.2. In certain embodiments, the L-tartrate salt of compound A13 is in a crystalline form having an X-ray powder diffractogram comprising peaks at two-theta angles (°) of approximately 8.6, 13.4, 14.3, 14.8, 16.7, 17.3, 17.7, 19.1, 20.0, 20.6, 21.2, 22.4, 22.8, 23.7, and 28.3.

In certain embodiments, a compound described herein is deuterium-enriched. In certain embodiments, a compound described herein is carbon-13 enriched. In certain embodiments, a compound described herein is carbon-14 enriched. In certain embodiments, a compound described herein contains one or more less prevalent isotopes for other elements, including, but not limited to, ⁵N for nitrogen; ¹⁷O or ¹⁸O for oxygen, and ³³S, ³⁴S, or ³⁶S for sulfur.

In certain embodiments, a compound described herein has an isotopic enrichment factor of no less than about 5, no less than about 10, no less than about 20, no less than about 30, no less than about 40, no less than about 50, no less than about 60, no less than about 70, no less than about 80, no less than about 90, no less than about 100, no less than about 200, no less than about 500, no less than about 1,000, no less than about 2,000, no less than about 5,000, or no less than about 10,000. In any events, however, an isotopic enrichment factor for a specified isotope is no greater than the maximum isotopic enrichment factor for the specified isotope, which is the isotopic enrichment factor when a compound at a given position is 100% enriched with the specified isotope. Thus, the maximum isotopic enrichment factor is different for different isotopes. The maximum isotopic enrichment factor is 6410 for deuterium and 90 for carbon-13.

In certain embodiments, a compound described herein has a deuterium enrichment factor of no less than about 64 (about 1% deuterium enrichment), no less than about 130 (about 2% deuterium enrichment), no less than about 320 (about 5% deuterium enrichment), no less than about 640 (about 10% deuterium enrichment), no less than about 1,300 (about 20% deuterium enrichment), no less than about 3,200 (about 50% deuterium enrichment), no less than about 4,800 (about 75% deuterium enrichment), no less than about 5,130 (about 80% deuterium enrichment), no less than about 5,450 (about 85% deuterium enrichment), no less than about 5,770 (about 90% deuterium enrichment), no less than about 6,090 (about 95% deuterium enrichment), no less than about 6,220 (about 97% deuterium enrichment), no less than about 6,280 (about 98% deuterium enrichment), no less than about 6,350 (about 99% deuterium enrichment), or no less than about 6,380 (about 99.5% deuterium enrichment). The deuterium enrichment can be determined using conventional analytical methods known to one of ordinary skill in the art, including mass spectrometry and nuclear magnetic resonance spectroscopy.

In certain embodiments, a compound described herein has a carbon-13 enrichment factor of no less than about 1.8 (about 2% carbon-13 enrichment), no less than about 4.5 (about 5% carbon-13 enrichment), no less than about 9 (about 10% carbon-13 enrichment), no less than about 18 (about 20% carbon-13 enrichment), no less than about 45 (about 50% carbon-13 enrichment), no less than about 68 (about 75% carbon-13 enrichment), no less than about 72 (about 80% carbon-13 enrichment), no less than about 77 (about 85% carbon-13 enrichment), no less than about 81 (about 90% carbon-13 enrichment), no less than about 86 (about 95% carbon-13 enrichment), no less than about 87 (about 97% carbon-13 enrichment), no less than about 88 (about 98% carbon-13 enrichment), no less than about 89 (about 99% carbon-13 enrichment), or no less than about 90 (about 99.5% carbon-13 enrichment). The carbon-13 enrichment can be determined using conventional analytical methods known to one of ordinary skill in the art, including mass spectrometry and nuclear magnetic resonance spectroscopy.

In certain embodiments, at least one of the atoms of a compound described herein, as specified as isotopically enriched, has isotopic enrichment of no less than about 1%, no less than about 2%, no less than about 5%, no less than about 10%, no less than about 20%, no less than about 50%, no less than about 70%, no less than about 80%, no less than about 90%, or no less than about 98%. In certain embodiments, the atoms of a compound described herein, as specified as isotopically enriched, have isotopic enrichment of no less than about 1%, no less than about 2%, no less than about 5%, no less than about 10%, no less than about 20%, no less than about 50%, no less than about 70%, no less than about 80%, no less than about 90%, or no less than about 98%. In any events, the isotopic enrichment of the isotopically enriched atom of a compound described herein is no less than the natural abundance of the isotope specified.

In certain embodiments, at least one of the atoms of a compound described herein, as specified as deuterium-enriched, has deuterium enrichment of no less than about 1%, no less than about 2%, no less than about 5%, no less than about 10%, no less than about 20%, no less than about 50%, no less than about 70%, no less than about 80%, no less than about 90%, or no less than about 98%. In certain embodiments, the atoms of a compound described herein, as specified as deuterium-enriched, have deuterium enrichment of no less than about 1%, no less than about 2%, no less than about 5%, no less than about 10%, no less than about 20%, no less than about 50%, no less than about 70%, no less than about 80%, no less than about 90%, or no less than about 98%.

In certain embodiments, at least one of the atoms of a compound described herein, as specified as ¹³C-enriched, has carbon-13 enrichment of no less than about 2%, no less than about 5%, no less than about 10%, no less than about 20%, no less than about 50%, no less than about 70%, no less than about 80%, no less than about 90%, or no less than about 98%. In certain embodiments, the atoms of a compound described herein, as specified as ¹³C-enriched, have carbon-13 enrichment of no less than about 1%, no less than about 2%, no less than about 5%, no less than about 10%, no less than about 20%, no less than about 50%, no less than about 70%, no less than about 80%, no less than about 90%, or no less than about 98%.

In certain embodiments, a compound described herein is isolated or purified. In certain embodiments, a compound described herein has a purity of at least about 50%, at least about 70%, at least about 80%, at least about 90%, at least about 95%, at least about 98%, at least about 99%, or at least about 99.5% by weight.

The compounds described herein are intended to encompass all possible stereoisomers unless a particular stereochemistry is specified. Where a compound described herein contains an alkenyl group, the compound may exist as one or mixture of geometric cis/trans (or 7E) isomers. Where structural isomers are interconvertible, the compound may exist as a single tautomer or a mixture of tautomers. This can take the form of proton tautomerism in the compound that contains, for example, an imino, keto, or oxime group; or so-called valence tautomerism in the compound that contain an aromatic moiety. It follows that a single compound may exhibit more than one type of isomerism.

A compound described herein can be enantiomerically pure, such as a ingle enantiomer or a single diastereomer, or be stereoisomeric mixtures, such as a mixture of enantiomers, e.g., a racemic mixture of two enantiomers; or a mixture of two or more diastereomers. As such, one of ordinary skill in the art will recognize that administration of a compound in its (R) form is equivalent, for compounds that undergo epimerization in vivo, to administration of the compound in its (S) form. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation.

When a compound described herein contains an acidic or basic moiety, it can also be provided as a pharmaceutically acceptable salt. See, Berge et al., J. Pharm. Sci. 1977, 66, 1-19; Handbook of Pharmaceutical Salts: Properties, Selection, and Use, 2nd ed.; Stahl and Wermuth Eds.; Wiley-VCH and VHCA, Zurich, 2011.

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (t)-DL-lactic acid, lactobionic acid, lauric acid, maleic acid, (−)-L-malic acid, malonic acid, (t)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid. In certain embodiments, a compound described herein is a hydrochloride salt. In certain embodiments, a compound described herein is a p-toluenesulfonate salt. In certain embodiments, a compound described herein is a di-p-toluenesulfonate salt.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, triethanolamine, trimethylamine, triethylamine, N-methyl-D-glucamine, 2-amino-2-(hydroxymethyl)-1,3-propanediol, and tromethamine.

A compound described herein may also be provided as a prodrug, which is a functional derivative of a compound, for example, of Formula I and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis.

The compounds described herein and crystalline forms thereof can be prepared, isolated, or obtained by any method known to one of ordinary skill in the art, for example, by following the procedures described in CN 106279160 and U.S. Pat. No. 10,906,901, the disclosure of each of which is incorporated herein by reference in its entirety.

### Pharmaceutical Compositions

In one embodiment, provided herein is a pharmaceutical composition, comprising a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; and a pharmaceutically acceptable excipient.

A pharmaceutical composition provided herein can be formulated in various dosage forms, including, but not limited to, dosage forms for oral, parenteral, and topical administration. The pharmaceutical composition can also be formulated as modified release dosage forms, including delayed-, extended-, prolonged-, sustained-, pulsatile-, controlled-, accelerated-, fast-, targeted-, programmed-release, and gastric retention dosage forms. These dosage forms can be prepared according to conventional methods and techniques known to those skilled in the art. See, e.g., Remington: The Science and Practice of Pharmacy, supra; Modified-Release Drug Delivery Technology, 2nd ed.; Rathbone et al., Eds.; Drugs and the Pharmaceutical Sciences 184; CRC Press: Boca Raton, F L, 2008.

In one embodiment, a pharmaceutical composition provided herein is formulated in a dosage form for oral administration. In another embodiment, a pharmaceutical composition provided herein is formulated in a dosage form for parenteral administration. In yet another embodiment, a pharmaceutical composition provided herein is formulated in a dosage form for intravenous administration. In yet another embodiment, a pharmaceutical composition provided herein is formulated in a dosage form for intramuscular administration. In yet another embodiment, a pharmaceutical composition provided herein is formulated in a dosage form for subcutaneous administration. In still another embodiment, a pharmaceutical composition provided herein is formulated in a dosage form for topical administration.

A pharmaceutical composition provided herein can be provided in a unit-dosage form or multiple-dosage form. A unit-dosage form, as used herein, refers to physically discrete a unit suitable for administration to a subject, and packaged individually as is known in the art. Each unit-dose contains a predetermined quantity of an active ingredient(s) (e.g., a compound provided herein) sufficient to produce the desired therapeutic effect, in association with the required pharmaceutical excipient(s). Examples of a unit-dosage form include, but are not limited to, an ampoule, syringe, and individually packaged tablet and capsule. A unit-dosage form may be administered in fractions or multiples thereof. A multiple-dosage form is a plurality of identical unit-dosage forms packaged in a single container to be administered in a segregated unit-dosage form. Examples of a multiple-dosage form include, are not limited to, a vial, bottle of tablets or capsules, or bottle of pints or gallons.

A pharmaceutical composition provided herein can be administered at once or multiple times at intervals of time. It is understood that the precise dosage and duration of treatment may vary with the age, weight, and condition of the subject being treated, and may be determined empirically using known testing protocols or by extrapolation from in vivo or in vitro test or diagnostic data. It is further understood that for any particular individual, specific dosage regimens should be adjusted over time according to the subject's need and the professional judgment of the person administering or supervising the administration of the pharmaceutical composition.

In one embodiment, provided herein is a pharmaceutical composition, comprising N-(2-((2-(dimethylamino)ethyl)-(methyl)amino)-4-methoxy-5-((4-(8-methylimidazo[1,2-a]-pyridin-3-yl)pyrimidin-2-yl)amino)phenyl)acrylamide A13; or a tautomer, a mixture of two or more tautomers, or an isotopic variant thereof; or a pharmaceutically acceptable salt, solvate, hydrate, or prodrug thereof; and a pharmaceutically acceptable excipient.

In another embodiment, provided herein is a pharmaceutical composition, comprising N-(2-((2-(dimethylamino)ethyl)-(methyl)amino)-4-methoxy-5-((4-(8-methylimidazo[1,2-a]pyridin-3-yl)pyrimidin-2-yl)amino)phenyl)acrylamide A13 in a crystalline form; and a pharmaceutically acceptable excipient.

In yet another embodiment, provided herein is a pharmaceutical composition, comprising a pharmaceutically acceptable salt of N-(2-((2-(dimethylamino)ethyl)-(methyl)amino)-4-methoxy-5-((4-(8-methylimidazo[1,2-a]pyridin-3-yl)pyrimidin-2-yl)amino)phenyl)acrylamide A13; or a pharmaceutically acceptable solvate or hydrate thereof; and a pharmaceutically acceptable excipient.

### A. Oral Administration

The pharmaceutical composition provided herein for oral administration can be provided in solid, semisolid, or liquid dosage forms for oral administration. As used herein, oral administration also includes buccal, lingual, and sublingual administration. Suitable oral dosage forms include, but are not limited to, tablets, fastmelts, chewable tablets, capsules, pills, strips, troches, lozenges, pastilles, cachets, pellets, medicated chewing gum, bulk powders, effervescent or non-effervescent powders or granules, oral mists, solutions, emulsions, suspensions, wafers, sprinkles, elixirs, and syrups. In addition to the active ingredient(s), the pharmaceutical composition can contain one or more pharmaceutically acceptable carriers or excipients, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500®); gelatin; sugars, such as sucrose, glucose, dextrose, molasses, and lactose; natural and synthetic gums, such as acacia, alginic acid, alginates, extract of Irish moss, Panwar gum, Ghatti gum, mucilage of isabgol husks, carboxymethylcellulose, methylcellulose, polyvinylpyrrolidone (PVP), VEEGUM®, larch arabinogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); and microcrystalline celluloses, such as AVICEL® PH-101, AVICEL® PH-103, AVICEL® PH-105, and AVICEL® RC-581. Suitable fillers include, but are not limited to, talc, calcium carbonate, microcrystalline cellulose, powdered cellulose, dextrates, kaolin, mannitol, silicic acid, sorbitol, starch, and pre-gelatinized starch. The amount of a binder or filler in the pharmaceutical composition provided herein varies upon the type of formulation, and is readily discernible to those of ordinary skill in the art. The binder or filler may be present from about 50 to about 99% by weight in the pharmaceutical composition provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets. The amount of a diluent in the pharmaceutical composition provided herein varies upon the type of formulation, and is readily discernible to those of ordinary skill in the art.

Suitable disintegrants include, but are not limited to, agar; bentonite; celluloses, such as methylcellulose and carboxymethylcellulose; wood products; natural sponge; cation-exchange resins; alginic acid; gums, such as guar gum and VEEGUM® HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; and algins. The amount of a disintegrant in the pharmaceutical composition provided herein varies upon the type of formulation, and is readily discernible to those of ordinary skill in the art. The pharmaceutical composition provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, such as peanut oil, cottonseed oil, sunflower oil, sesame oil, olive oil, corn oil, and soybean oil; zinc stearate; ethyl oleate; ethyl laureate; agar; starch; lycopodium; and silica or silica gels, such as AEROSIL® 200 and CAB-O-SIL®. The amount of a lubricant in the pharmaceutical composition provided herein varies upon the type of formulation, and is readily discernible to those of ordinary skill in the art. The pharmaceutical compositions provided herein may contain about 0.1 to about 5% by weight of a lubricant.

Suitable glidants include, but are not limited to, colloidal silicon dioxide, CAB-O-SIL®, and asbestos-free talc. Suitable coloring agents include, but are not limited to, any of the approved, certified, water soluble FD&C dyes, and water insoluble FD&C dyes suspended on alumina hydrate, and color lakes. A color lake is a combination by adsorption of a water-soluble dye to a hydrous oxide of a heavy metal, resulting in an insoluble form of the dye. Suitable flavoring agents include, but are not limited to, natural flavors extracted from plants, such as fruits, and synthetic blends of compounds which produce a pleasant taste sensation, such as peppermint and methyl salicylate. Suitable sweetening agents include, but are not limited to, sucrose, lactose, mannitol, syrups, glycerin, and artificial sweeteners, such as saccharin and aspartame. Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suitable suspending and dispersing agents include, but are not limited to, sodium carboxymethylcellulose, pectin, tragacanth, VEEGUM®, acacia, sodium carboxymethylcellulose, hydroxypropyl methylcellulose, and polyvinylpyrrolidone. Suitable preservatives include, but are not limited to, glycerin, methyl and propylparaben, benzoic add, and sodium benzoate and alcohol. Suitable wetting agents include, but are not limited to, propylene glycol monostearate, sorbitan monooleate, diethylene glycol monolaurate, and polyoxyethylene lauryl ether. Suitable solvents include, but are not limited to, glycerin, sorbitol, ethyl alcohol, and syrup. Suitable non-aqueous liquids utilized in emulsions include, but are not limited to, mineral oil and cottonseed oil. Suitable organic acids include, but are not limited to, citric and tartaric acid. Suitable sources of carbon dioxide include, but are not limited to, sodium bicarbonate and sodium carbonate.

It should be understood that many carriers and excipients may serve several functions, even within the same formulation.

The pharmaceutical composition provided herein for oral administration can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredient(s) from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms can be prepared from an active ingredient(s) in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical composition provided herein for oral administration can be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient(s). The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient(s).

The pharmaceutical composition provided herein for oral administration can be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquid or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing an active ingredient(s), and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These dosage forms can further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical composition provided herein for oral administration can be also provided in the forms of liposomes, micelles, microspheres, or nanosystems. Micellar dosage forms can be prepared as described in U.S. Pat. No. 6,350,458.

The pharmaceutical composition provided herein for oral administration can be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all the dosage forms described herein.

The pharmaceutical composition provided herein for oral administration can be formulated as immediate or modified release dosage forms, including delayed-, sustained, pulsed-, controlled, targeted-, and programmed-release forms.

### B. Parenteral Administration

The pharmaceutical composition provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, intravesical, and subcutaneous administration.

The pharmaceutical composition provided herein for parenteral administration can be formulated in any dosage forms that are suitable for parenteral administration, including, but not limited to, solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science. See, e.g., Remington: The Science and Practice of Pharmacy, supra.

The pharmaceutical composition provided herein for parenteral administration can include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringer's injection, isotonic dextrose injection, sterile water injection, dextrose and lactated Ringer's injection. Suitable non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Suitable water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants include those described herein, such as bisulfite and sodium metabisulfite. Suitable local anesthetics include, but are not limited to, procaine hydrochloride. Suitable suspending and dispersing agents include those described herein, such as sodium carboxymethylcellulose, hydroxypropyl methylcellulose, and polyvinylpyrrolidone. Suitable emulsifying agents include those described herein, such as polyoxyethylene sorbitan monolaurate, polyoxyethylene sorbitan monooleate 80, and triethanolamine oleate. Suitable sequestering or chelating agents include, but are not limited to, EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®).

When the pharmaceutical composition provided herein is formulated for multiple dosage administration, multiple dosage parenteral formulations must contain an antimicrobial agent at bacteriostatic or fungistatic concentrations. All parenteral formulations must be sterile, as known and practiced in the art.

In one embodiment, the pharmaceutical composition for parenteral administration is provided as a ready-to-use sterile solution. In another embodiment, the pharmaceutical composition is provided as a sterile dry soluble product, including a lyophilized powder and hypodermic tablet, to be reconstituted with a vehicle prior to use. In yet another embodiment, the pharmaceutical composition is provided as a ready-to-use sterile suspension. In yet another embodiment, the pharmaceutical composition is provided as a sterile dry insoluble product to be reconstituted with a vehicle prior to use. In still another embodiment, the pharmaceutical composition is provided as a ready-to-use sterile emulsion.

The pharmaceutical composition provided herein for parenteral administration can be formulated as immediate or modified release dosage forms, including delayed-, sustained, pulsed-, controlled, targeted-, and programmed-release forms.

The pharmaceutical composition provided herein for parenteral administration can be formulated as a suspension, solid, semi-solid, or thixotropic liquid, for administration as an implanted depot. In one embodiment, the pharmaceutical composition provided herein are dispersed in a solid inner matrix, which is surrounded by an outer polymeric membrane that is insoluble in body fluids but allows the active ingredient(s) in the pharmaceutical composition to diffuse through.

Suitable inner matrixes include, but are not limited to, polymethylmethacrylate, polybutylmethacrylate, plasticized or unplasticized polyvinylchloride, plasticized nylon, plasticized polyethylene terephthalate, natural rubber, polyisoprene, polyisobutylene, polybutadiene, polyethylene, ethylene-vinyl acetate copolymers, silicone rubbers, polydimethylsiloxanes, silicone carbonate copolymers, hydrophilic polymers (such as hydrogels of esters of acrylic and methacrylic acid), collagen, cross-linked polyvinyl alcohol, and cross-linked partially hydrolyzed polyvinyl acetate.

Suitable outer polymeric membranes include, but are not limited to, polyethylene, polypropylene, ethylene/propylene copolymers, ethylene/ethyl acrylate copolymers, ethylene/vinyl acetate copolymers, silicone rubbers, polydimethylsiloxanes, neoprene rubber, chlorinated polyethylene, polyvinylchloride, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### C. Topical Administration

The pharmaceutical composition provided herein can be administered topically to the skin, orifices, or mucosa. The topical administration, as used herein, includes (intra)dermal, conjunctival, intracorneal, intraocular, ophthalmic, auricular, transdermal, nasal, vaginal, urethral, respiratory, and rectal administration.

The pharmaceutical composition provided herein can be formulated in any dosage forms that are suitable for topical administration for local or systemic effect, including, but not limited to, emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, and dermal patches. The topical formulations of the pharmaceutical composition provided herein can also comprise liposomes, micelles, microspheres, and nanosystems.

Pharmaceutically acceptable carriers and excipients suitable for use in the topical formulations include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The pharmaceutical composition can also be administered topically by electroporation, iontophoresis, phonophoresis, sonophoresis, or microneedle or needle-free injection, such as POWDERJECT™ and BIOJECT™.

The pharmaceutical composition provided herein can be provided in the forms of ointments, creams, and gels. Suitable ointment vehicles include oleaginous or hydrocarbon vehicles, including lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid. See, e.g., Remington: The Science and Practice of Pharmacy, supra. These vehicles are emollient but generally require addition of antioxidants and preservatives.

Suitable cream base can be oil-in-water or water-in-oil. Suitable cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include, but are not limited to, crosslinked acrylic acid polymers, such as carbomers, carboxypolyalkylenes, and CARBOPOL®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical composition provided herein can be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with an active ingredient(s); and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, and hydrogels, such as polyvinyl alcohol, hydroxyethyl methacrylate, and polyacrylic acid. Combinations of the various vehicles can also be used. Rectal and vaginal suppositories may be prepared by compressing or molding. The typical weight of a rectal and vaginal suppository is about 2 to about 3 g.

The pharmaceutical composition provided herein can be administered ophthalmically in the forms of solutions, suspensions, ointments, emulsions, gel-forming solutions, powders for solutions, gels, ocular inserts, and implants.

The pharmaceutical composition provided herein can be administered intranasally or by inhalation to the respiratory tract. The pharmaceutical composition can be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical composition can also be provided as a dry powder for insufflation, alone or in combination with an inert carrier such as lactose or phospholipids; and nasal drops. For intranasal use, the powder can comprise a bioadhesive agent, including chitosan or cyclodextrin.

Solutions or suspensions for use in a pressurized container, pump, spray, atomizer, or nebulizer can be formulated to contain ethanol, aqueous ethanol, or a suitable alternative agent for dispersing, solubilizing, or extending release of an active ingredient(s); a propellant as solvent; and/or a surfactant, such as sorbitan trioleate, oleic acid, or an oligolactic acid.

The pharmaceutical composition provided herein can be micronized to a size suitable for delivery by inhalation, such as about 50 micrometers or less, or about 10 micrometers or less. Particles of such sizes can be prepared using a comminuting method known to those skilled in the art, such as spiral jet milling, fluid bed jet milling, supercritical fluid processing to form nanoparticles, high pressure homogenization, or spray drying.

Capsules, blisters, and cartridges for use in an inhaler or insufflator can be formulated to contain a powder mix of the pharmaceutical composition provided herein; a suitable powder base, such as lactose or starch; and a performance modifier, such as 1-leucine, mannitol, or magnesium stearate. The lactose may be anhydrous or in the form of the monohydrate. Other suitable excipients or carriers include, but are not limited to, dextran, glucose, maltose, sorbitol, xylitol, fructose, sucrose, and trehalose. The pharmaceutical composition provided herein for inhaled/intranasal administration can further comprise a suitable flavor, such as menthol and levomenthol; and/or sweeteners, such as saccharin and saccharin sodium.

The pharmaceutical composition provided herein for topical administration can be formulated to be immediate release or modified release, including delayed-, sustained-, pulsed-, controlled-, targeted, and programmed release.

### D. Modified Release

The pharmaceutical composition provided herein can be formulated as a modified release dosage form. As used herein, the term “modified release” refers to a dosage form in which the rate or place of release of an active ingredient(s) is different from that of an immediate dosage form when administered by the same route. Modified release dosage forms include, but are not limited to, delayed-, extended-, prolonged-, sustained-, pulsatile-, controlled-, accelerated- and fast-, targeted-, programmed-release, and gastric retention dosage forms. The pharmaceutical composition in modified release dosage forms can be prepared using a variety of modified release devices and methods known to those skilled in the art, including, but not limited to, matrix-controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphism of the active ingredient(s).

### 1. Matrix Controlled Release Devices

The pharmaceutical composition provided herein in a modified release dosage form can be fabricated using a matrix-controlled release device known to those skilled in the art. See, e.g., Takada et al. in Encyclopedia of Controlled Drug Delivery, Mathiowitz Ed.; Wiley, 1999; Vol. 2.

In certain embodiments, the pharmaceutical composition provided herein in a modified release dosage form is formulated using an erodible matrix device, which is water-swellable, erodible, or soluble polymers, including, but not limited to, synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum Ghatti, guar gum, xanthan gum, and scleroglucan; starches, such as dextrin and maltodextrin; hydrophilic colloids, such as pectin; phosphatides, such as lecithin; alginates; propylene glycol alginate; gelatin-collagen; cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethyl hydroxyethyl cellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methyl methacrylate, ethyl methacrylate, ethylacrylate, (2-dimethylaminoethyl)methacrylate, and (trimethylaminoethyl)methacrylate chloride.

In certain embodiments, the pharmaceutical composition provided herein is formulated with a non-erodible matrix device. The active ingredient(s) is dissolved or dispersed in an inert matrix and is released primarily by diffusion through the inert matrix once administered. Materials suitable for use as a non-erodible matrix device include, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinyl acetate copolymers, ethylene/propylene copolymers, ethylene/ethyl acrylate copolymers, vinyl chloride copolymers with vinyl acetate, vinylidene chloride, ethylene and propylene, ionomer polyethylene terephthalate, butyl rubbers, epichlorohydrin rubbers, ethylene/vinyl alcohol copolymer, ethylene/vinyl acetate/vinyl alcohol terpolymer, ethylene/vinyloxyethanol copolymer, polyvinyl chloride, plasticized nylon, plasticized polyethylene terephthalate, natural rubber, silicone rubbers, polydimethylsiloxanes, and silicone carbonate copolymers; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix-controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

The pharmaceutical composition provided herein in a modified release dosage form can be prepared by methods known to those skilled in the art, including direct compression, dry or wet granulation followed by compression, and melt-granulation followed by compression.

### 2. Osmotic Controlled Release Devices

The pharmaceutical composition provided herein in a modified release dosage form can be fabricated using an osmotic controlled release device, including, but not limited to, one-chamber system, two-chamber system, asymmetric membrane technology (AMT), and extruding core system (ECS). In general, such devices have at least two components: (a) a core which contains an active ingredient; and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents is water-swellable hydrophilic polymers, which are also referred to as “osmopolymers” and “hydrogels.” Suitable water-swellable hydrophilic polymers as osmotic agents include, but are not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents is osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates can be employed to influence how rapidly the active ingredient(s) is initially delivered from the dosage form. For example, amorphous sugars, such as MANNOGEM™ EZ can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core can also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane can also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane can be formed post-coaling by mechanical or laser drilling. Delivery port(s) can also be formed in situ by erosion of a plug of water-soluble material or by rupture of a thinner portion of the membrane over an indentation in the core. In addition, delivery ports can be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical composition in an osmotic controlled-release dosage form can further comprise additional conventional excipients or carriers as described herein to promote performance or processing of the formulation.

The osmotic controlled-release dosage forms can be prepared according to conventional methods and techniques known to those skilled in the art. See, e.g., Remington: The Science and Practice of Pharmacy, supra; Santus and Baker, J. Controlled Release, 1995, 35, 1-21; Verma et al., Drug Dev. Ind Pharm., 2000, 26, 695-708; Verma et al., J. Controlled Release, 2002, 79, 7-27.

In certain embodiments, the pharmaceutical composition provided herein is formulated as an AMT controlled-release dosage form, which comprises an asymmetric osmotic membrane that coats a core comprising the active ingredient(s) and other pharmaceutically acceptable excipients or carriers. See, e.g., U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coaling method.

In certain embodiments, the pharmaceutical composition provided herein is formulated as an ESC controlled-release dosage form, which comprises an osmotic membrane that coats a core comprising the active ingredient(s), a hydroxyethyl cellulose, and other pharmaceutically acceptable excipients or carriers.

### 3. Multiparticulate Controlled Release Devices

The pharmaceutical composition provided herein in a modified release dosage form can be fabricated as a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates can be made by the processes known to those skilled in the art, including wet- and dry-granulation, extrusion/spheronization, roller-compaction, melt-congealing, and by spray-coating seed cores. See, e.g., Multiparticulate Oral Drug Delivery; Ghebro-Sellassie Eds.; Drugs and the Pharmaceutical Sciences 65; CRC Press: 1994; and Pharmaceutical Palletization Technology; Ghebre-Selassie Eds.; Drugs and the Pharmaceutical Sciences 37; CRC Press: 1989.

Other excipients or carriers as described herein can be blended with the pharmaceutical composition to aid in processing and forming the multipartiulates. The resulting particles can themselves constitute the multiparticulate device or can be coated by various film-forming materials, such as enteric polymers, water-swellable, and water-soluble polymers. The multiparticulates can be further processed as a capsule or a tablet.

### 4. Targeted Delivery

The pharmaceutical composition provided herein can also be formulated to be targeted to a particular tissue, receptor, or other area of the body of the subject to be treated, including liposome-, resealed erythrocyte-, and antibody-based delivery systems. Examples include, but are not limited to, those disclosed in U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874.

### Methods of Treatment

In one embodiment, provided herein is method of treating, preventing, or ameliorating one or more symptoms of lung cancer bearing an EGFR mutation in a subject, comprising administering to the subject in need thereof a therapeutically effective amount of a compound described herein, e.g., a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In another embodiment, provided herein is method of treating, preventing, or ameliorating one or more symptoms of lung cancer in a subject, comprising the steps of:
(c) determining the presence of an EGFR exon 20 mutation in a sample from the subject; and
(d) if the sample is determined to have an EGFR exon 20 mutation, administering to the subject a therapeutically effective amount of a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In certain embodiments, the lung cancer is localized. In certain embodiments, the lung cancer is regional. In certain embodiments, the lung cancer is distant. In certain embodiments, the lung cancer is advanced. In certain embodiments, the lung cancer is locally advanced. In certain embodiments, the lung cancer is unresectable. In certain embodiments, the lung cancer is inoperable. In certain embodiments, the lung cancer is incurable. In certain embodiments, the lung cancer is metastatic. In certain embodiments, the lung cancer is recurrent. In certain embodiments, the lung cancer is relapsed. In certain embodiments, the lung cancer is refractory. In certain embodiments, the lung cancer is refractory to a standard therapy. In certain embodiments, the lung cancer is intolerant of a standard therapy. In certain embodiments, the lung cancer is drug-resistant. In certain embodiments, the lung cancer is resistant to chemotherapy. In certain embodiments, the lung cancer is resistant to a targeted drug therapy. In certain embodiments, the lung cancer is resistant to a tyrosine kinase inhibitor (TKI). In certain embodiments, the lung cancer is resistant to an EGFR inhibitor. In certain embodiments, the lung cancer is resistant to a first-generation EGFR inhibitor. In certain embodiments, the lung cancer is resistant to a second-generation EGFR inhibitor. In certain embodiments, the lung cancer is resistant to a third-generation EGFR inhibitor. In certain embodiments, the lung cancer is resistant to afatinib, brigatinib, dacomitinib, erlotinib, gefitinib, icotinib, or osimertinib.

In certain embodiments, the lung cancer is stage L In certain embodiments, the lung cancer is stage IA1, IA2, IA3, or IB. In certain embodiments, the lung cancer is stage IL In certain embodiments, the lung cancer is stage IIA or IB. In certain embodiments, the lung cancer is stage III. In certain embodiments, the lung cancer is stage IIIA, IIB, or IIIC. In certain embodiments, the lung cancer is stage IV. In certain embodiments, the lung cancer is stage IVA or IVB. In certain embodiments, the lung cancer is stage II, III, or IV. In certain embodiments, the lung cancer is stage III or IV.

In certain embodiments, the lung cancer is small cell lung cancer. In certain embodiments, the lung cancer is non-small cell lung cancer (NSCLC). In certain embodiments, the lung cancer is squamous cell carcinoma, adenocarcinoma, or large cell carcinoma.

In certain embodiments, NSCLC is localized. In certain embodiments, NSCLC is regional. In certain embodiments, NSCLC is distant. In certain embodiments, NSCLC is advanced. In certain embodiments, NSCLC is locally advanced. In certain embodiments, NSCLC is unresectable. In certain embodiments, NSCLC is inoperable. In certain embodiments, NSCLC is incurable. In certain embodiments, NSCLC is metastatic. In certain embodiments, NSCLC is recurrent. In certain embodiments, NSCLC is relapsed. In certain embodiments, NSCLC is refractory. In certain embodiments, NSCLC is refractory to a standard therapy. In certain embodiments, NSCLC is intolerant of a standard therapy. In certain embodiments, NSCLC is drug-resistant. In certain embodiments, NSCLC is resistant to chemotherapy. In certain embodiments, NSCLC is resistant to a targeted drug therapy. In certain embodiments, NSCLC is resistant to a TKI. In certain embodiments, NSCLC is resistant to an EGFR inhibitor. In certain embodiments, NSCLC is resistant to a first-generation EGFR inhibitor. In certain embodiments, NSCLC is resistant to a second-generation EGFR inhibitor. In certain embodiments, NSCLC is resistant to a third-generation EGFR inhibitor. In certain embodiments, NSCLC is resistant to afatinib, brigatinib, dacomitinib, erlotinib, gefitinib, icotinib, or osimertinib.

In certain embodiments, NSCLC is stage L In certain embodiments, NSCLC is stage IA1, IA2, IA3, or LB. In certain embodiments, NSCLC is stage IL In certain embodiments, NSCLC is stage IIA or IB. In certain embodiments, NSCLC is stage IL In certain embodiments, NSCLC is stage LA, IIB, or MC. In certain embodiments, NSCLC is stage V. In certain embodiments, NSCLC is stage WA or WB. In certain embodiments, NSCLC is stage II, M, or V. In certain embodiments, NSCLC is stage M or V.

In certain embodiments, NSCLC harbors an EGFR mutation. In certain embodiments, NSCLC harbors an EGFR mutation in exon 18. In certain embodiments, NSCLC harbors an EGFR point mutation in exon 18. In certain embodiments, NSCLC harbors G719X in exon 18. In certain embodiments, NSCLC harbors G719S. In certain embodiments, NSCLC harbors an EGFR exon 18 deletion. In certain embodiments, NSCLC harbors an EGFR exon 18 insertion.

In certain embodiments, NSCLC harbors an EGFR mutation in exon 19. In certain embodiments, NSCLC harbors an EGFR point mutation in exon 19. In certain embodiments, NSCLC harbors an EGFR exon 19 deletion. In certain embodiments, NSCLC harbors an EGFR exon 19 insertion.

In certain embodiments, NSCLC harbors an EGFR mutation in exon 20. In certain embodiments, NSCLC harbors an EGFR point mutation in exon 20. In certain embodiments, NSCLC harbors S768L In certain embodiments, NSCLC harbors T790M. In certain embodiments, NSCLC harbors S768I, V769L, H773Y, V774M, R776H, or R776C. In certain embodiments, NSCLC harbors an EGFR exon 20 deletion.

In certain embodiments, NSCLC harbors an EGFR exon 20 insertion. In certain embodiments, NSCLC harbors an EGFR exon 20 insertion between D761 and C775. In certain embodiments, NSCLC harbors an EGFR exon 20 insertion of 1, 2, 3, 4, 5, 6, or 7 amino acids between D761 and C775. In certain embodiments, NSCLC harbors an EGFR exon 20 insertion of one amino acid between D761 and C775. In certain embodiments, NSCLC harbors an EGFR exon 20 insertion of 2 amino acids between D761 and C775. In certain embodiments, NSCLC harbors an EGFR exon 20 insertion of 3 amino acids between D761 and C775. In certain embodiments, NSCLC harbors an EGFR exon 20 insertion of 4 amino acids between D761 and C775. In certain embodiments, NSCLC harbors an EGFR exon 20 insertion of 5 amino acids between D761 and C775. In certain embodiments, NSCLC harbors an EGFR exon 20 insertion of 6 amino acids between D761 and C775. In certain embodiments, NSCLC harbors an EGFR exon 20 insertion of 7 amino acids between D761 and C775.

In certain embodiments, NSCLC harbors an EGFR exon 20 insertion of D761_E762insX, where X is an insertion of 1, 2, 3, 4, 5, 6, or 7 amino acids, each independently selected from the twenty natural amino acids. In certain embodiments, NSCLC harbors an EGFR exon 20 insertion of A763_Y764insX, where X is an insertion of 1, 2, 3, 4, 5, 6, or 7 amino acids, each independently selected from the twenty natural amino acids. In certain embodiments, NSCLC harbors an EGFR exon 20 insertion of Y764_V765insX, where X is an insertion of 1, 2, 3, 4, 5, 6, or 7 amino acids, each independently selected from the twenty natural amino acids. In certain embodiments, NSCLC harbors an EGFR exon 20 insertion of V765_-766insX, where X is an insertion of 1, 2, 3, 4, 5, 6, or 7 amino acids, each independently selected from the twenty natural amino acids. In certain embodiments, NSCLC harbors an EGFR exon 20 insertion of A767_S768insX, where X is an insertion of 1, 2, 3, 4, 5, 6, or 7 amino acids, each independently selected from the twenty natural amino acids. In certain embodiments, NSCLC harbors an EGFR exon 20 insertion of S768_V769insX, where X is an insertion of 1, 2, 3, 4, 5, 6, or 7 amino acids, each independently selected from the twenty natural amino acids. In certain embodiments, NSCLC harbors an EGFR exon 20 insertion of V769_D770insX, where X is an insertion of 1, 2, 3, 4, 5, 6, or 7 amino acids, each independently selected from the twenty natural amino acids. In certain embodiments, NSCLC harbors an EGFR exon 20 insertion of D770_N771insX, where X is an insertion of 1, 2, 3, 4, 5, 6, or 7 amino acids, each independently selected from the twenty natural amino acids. In certain embodiments, NSCLC harbors an EGFR exon 20 insertion of N771_P772insX, where X is an insertion of 1, 2, 3, 4, 5, 6, or 7 amino acids, each independently selected from the twenty natural amino acids. In certain embodiments, NSCLC harbors an EGFR exon 20 insertion of P772_H773insX, where X is an insertion of 1, 2, 3, 4, 5, 6, or 7 amino acids, each independently selected from the twenty natural amino acids. In certain embodiments, NSCLC harbors an EGFR exon 20 insertion of H773_V774insX, where X is an insertion of 1, 2, 3, 4, 5, 6, or 7 amino acids, each independently selected from the twenty natural amino acids. In certain embodiments, NSCLC harbors an EGFR exon 20 insertion of V774_C775insX, where X is an insertion of 1, 2, 3, 4, 5, 6, or 7 amino acids, each independently selected from the twenty natural amino acids.

In certain embodiments, NSCLC harbors an EGFR exon 20 insertion of A763_Y764insFQEA, A763_Y764insLQEA, A767_S768insASV, A767_S768insTLA, S768_D770dup, V769_D770insGE, V769_D770insASV, V769_D770insGSV, V769_D770insGVV, V769_D770insMASVD, D770_N771insG, D770_N771insY, D770N771insGV, D770N771insGT, D770N771insNPG, D770N771insSVD, D770_N771delinsGY, N771dup, N771_F772insH, N771_H772insN, N771_P772insHH, N771_P772insSVDNR, N771delinsFR, N771delinsGY, N771_H773dup, P772_H773insNP, P772_H773insDNP, P772_H773insNPH, H773_V774insAH, H773_V774insH, H773_V774insY, H773_V774insAH, H773_V774insPH, H773_V774insNPH, or V774_C775insHV. In certain embodiments, NSCLC harbors A763_Y764insFQEA, A767_S768insASV, S768_D770dup, V769_D770insASV, D770_N771insNPG, D770N771insSVD, D770N771delinsGY, D770N771delinsFH, N771P772delinsFH, N771_N773dup, or H773_V774insNPH. In certain embodiments, NSCLC harbors A763_Y764insFQEA, V769_D770insGE, V769_D770insASV, D770_N771insNPG, D770_N771insSVD, or H773_V774insNPH. In certain embodiments, NSCLC harbors A763_Y764insFQEA. In certain embodiments, NSCLC harbors V769_D770insGE. In certain embodiments, NSCLC harbors V769_D770insASV. In certain embodiments, NSCLC harbors D770N771insNPG. In certain embodiments, NSCLC harbors D770_N771insSVD. In certain embodiments, NSCLC harbors H773_V774insNPH.

As used herein, the EGFR mutations are named according to Dumnen and Antonarakis, Hum. Mutat. 2000, 15, 7-12, the disclosure of which is incorporated herein by reference in its entirety. For example, S768_D770dup represents the duplication of the amino acid sequence from residues S768 to D770; D770_N771delinsGY represents the deletion of residues D770 and N771 and an insertion of GY in the same place; and A763_Y764insFQEA represents an insertion of FQEA between residues A763 and Y764.

In certain embodiments, NSCLC harbors an EGFR mutation in exon 21. In certain embodiments, NSCLC harbors an EGFR point mutation in exon 21. In certain embodiments, NSCLC harbors L858R in exon 21. In certain embodiments, NSCLC harbors L861Q in exon 21. In certain embodiments, NSCLC harbors an EGFR exon 21 deletion. In certain embodiments, NSCLC harbors an EGFR exon 21 insertion.

In certain embodiments, NSCLC harbors an EGFR mutation in exon 22. In certain embodiments, NSCLC harbors an EGFR point mutation in exon 22. In certain embodiments, NSCLC harbors an EGFR exon 22 deletion. In certain embodiments, NSCLC harbors an EGFR exon 22 insertion.

In certain embodiments, the sample is a bodily fluid. In certain embodiments, the sample is a tissue. In certain embodiments, the sample is a lung cancer specimen. In certain embodiments, the sample is a biopsy. In certain embodiments, the sample is a lung cancer biopsy.

In certain embodiments, an EGFR mutation is determined by nucleic acid sequencing. In certain embodiments, an EGFR mutation is determined using fragment analysis. In certain embodiments, an EGFR mutation is determined by Sanger sequencing. In certain embodiments, an EGFR mutation is detected using a method described in Naidoo et al., Cancer 2015, 121, 3212-20, the disclosure of which is incorporated herein by reference in its entirety.

In certain embodiments, the subject is untreated, that is, has not received a therapy for the lung cancer. In certain embodiments, the subject is treated for the lung cancer. In certain embodiments, the subject has failed a prior therapy. In certain embodiments, the subject has failed more than one prior therapy.

In certain embodiments, the subject is a mammal. In certain embodiments, the subject is a human. In certain embodiments, the subject is an adult human. In certain embodiments, the subject is a pediatric human.

A method provided herein encompasses treating a subject regardless of patient's age, although some diseases are more common in certain age groups.

In certain embodiments, the therapeutically effective amount of a compound described herein, e.g., compound A13, is ranging from about 0.1 to about 100 mg/kg per day, from about 0.2 to about 50 mg/kg per day, from about 0.5 to about 20 mg/kg per day, or from about 1 to about 10 mg/kg per day. In one embodiment, the therapeutically effective amount of a compound described herein, e.g., compound A13, is ranging from about 0.1 to about 100 mg/kg per day. In another embodiment, the therapeutically effective amount of a compound described herein, e.g., compound A13, is ranging from about 0.2 to about 50 mg/kg per day. In yet another embodiment, the therapeutically effective amount of a compound described herein, e.g., compound A13, is ranging from about 0.5 to about 20 mg/kg per day. In yet another embodiment, the therapeutically effective amount of a compound described herein, e.g., compound A13, is ranging from about 1 to about 10 mg/kg per day. In still another embodiment, the therapeutically effective amount of a compound described herein, e.g., compound A22, is about 0.5, about 0.6, about 0.7, about 0.8, about 0.9, about 1, about 2, about 3, about 4, about 5, about 7, about 8, about 9, or about 10 mg/kg per day.

In certain embodiments, the therapeutically effective amount of a compound described herein, e.g., compound A13, is ranging from about 10 to about 1,000 mg per day, from about 20 to about 500 mg per day, or from about 50 to about 200 mg per day. In one embodiment, the therapeutically effective amount of a compound described herein, e.g., compound A13, is ranging from about 10 to about 1,000 mg per day. In another embodiment, the therapeutically effective amount of a compound described herein, e.g., compound A13, is ranging from about 20 to about 500 mg per day. In yet another embodiment, the therapeutically effective amount of a compound described herein, e.g., compound A13, is ranging from about 50 to about 200 mg per day. In yet another embodiment, the therapeutically effective amount of a compound described herein, e.g., compound A13, is about 50, about 100, about 150, about 200, about 250, about 300, about 350, about 400, about 450, or about 500 mg per day.

It is understood that the administered dose of a compound described herein can also be expressed in units other than mg/kg every other day. For example, doses for parenteral administration can be expressed as mg/m² per day. One of ordinary skill in the art would readily know how to convert doses from mg/kg per day to mg/m² per day to given either the height or weight of a subject or both. For example, a dose of 1 mg/m² per day for a 65 kg human is approximately equal to 58 mg/kg per day.

Depending on the disease to be treated and the subject's condition, a compound described herein may be administered by oral, parenteral (e.g., intramuscular, intraperitoneal, intravenous, CIV, intracisternal injection or infusion, subcutaneous injection, or implant), inhalation, nasal, vaginal, rectal, sublingual, or topical (e.g., transdermal or local) routes of administration.

In one embodiment, a compound described herein, e.g., compound A13, is administered orally. In another embodiment, a compound described herein, e.g., compound A13, is administered parenterally. In yet another embodiment, a compound described herein, e.g., compound A13, is administered intravenously. In yet another embodiment, a compound described herein, e.g., compound A13, is administered intramuscularly. In yet another embodiment, a compound described herein, e.g., compound A13, is administered subcutaneously. In still another embodiment, a compound described herein, e.g., compound A13, is administered topically.

A compound described herein, e.g., compound A13, can be delivered as a single dose such as, e.g., a single bolus injection, or oral tablets or pills; or over time such as, e.g., continuous infusion over time or divided bolus doses over time. A compound described herein, e.g., compound A13, can be administered repetitively, if necessary, for example, until the subject experiences stable disease or regression, or until the subject experiences disease progression or unacceptable toxicity. Stable disease or lack thereof is determined by a method known in the art such as evaluation of subject's symptoms, physical examination, visualization of the cancer that has been imaged using X-ray, CAT, PET, or MRI scan and other commonly accepted evaluation modalities.

A compound described herein, e.g., compound A13, can be administered once daily (QD), or divided into multiple daily doses such as twice daily (BID), and three times daily (TID). In addition, the administration can be continuous, i.e., every day, or intermittently. The term “intermittent” or “intermittently” as used herein is intended to mean stopping and starting at either regular or irregular intervals. For example, intermittent administration of a compound described herein, e.g., compound A13, is administration for one to six days per week, administration in cycles (e.g., daily administration for two to eight consecutive weeks, then a rest period with no administration for up to one week), or administration on alternate days.

It will be understood, however, that the specific dose level and frequency of dosage for any particular subject can be varied and will depend upon a variety of factors including the activity of the specific compound employed, e.g., compound A13, the metabolic stability and length of action of the compound, the age, body weight, general health, sex, diet, mode and time of administration, rate of excretion, drug combination, the severity of the particular condition, and the host undergoing therapy.

In certain embodiments, a compound described herein, e.g., compound A13, is cyclically administered to a subject to be treated. Cycling therapy involves the administration of the compound for a period of time, followed by a rest for a period of time, and repeating this sequential administration. Cycling therapy can reduce the development of resistance to one or more of the therapies, avoid or reduce the side effects of one of the therapies, and/or improves the efficacy of the treatment.

Consequently, in one embodiment, a compound described herein, e.g., compound A13, is administered for a cycle of about one week, about two weeks, about three weeks, about four weeks, about five weeks, about six weeks, about eight weeks, or about ten weeks, with a rest period of about 1 day to about four weeks. In one embodiment, a compound described herein, e.g., compound A13, is administered for a cycle of three weeks, four weeks, five weeks, or six weeks with a rest period of 1, 3, 5, 7, 9, 12, or 14 days. In certain embodiments, the rest period is 7 days. In certain embodiments, the rest period is 14 days. In certain embodiments, the rest period is a period that is sufficient for bone marrow recovery. The frequency, number, and length of dosing cycles can be increased or decreased.

In one embodiment, a compound described herein, e.g., compound A13, is administered for three weeks in a 28-day cycle with a 7-day rest period. In one embodiment, in a 28-day cycle with a 7-day rest period, a compound described herein, e.g., compound A13, is administered every day of a week.

In certain embodiments, the subject is treated with a compound described herein, e.g., compound A13, from about 1 to about 50, from about 2 to about 20, from about 2 to 10, or from about 4 to about 8 cycles. In certain embodiments, the subject is treated with a compound described herein, e.g., compound A13, from about 1 to about 50 cycles. In certain embodiments, the subject is treated with a compound described herein, e.g., compound A13, from about 2 to about 20 cycles. In certain embodiments, the subject is treated with a compound described herein, e.g., compound A13, from about 2 to 10 cycles. In certain embodiments, the subject is treated with a compound described herein, e.g., compound A13, from about 4 to about 8 cycles.

In one embodiment, provided herein is a method of inhibiting the growth of a lung cancer cell, comprising contacting the cell with an effective amount of a compound described herein, e.g., a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

The disclosure will be further understood by the following non-limiting examples.

### EXAMPLES

As used herein, the symbols and conventions used in these processes, schemes, and examples, regardless of whether a particular abbreviation is specifically defined, are consistent with those used in the contemporary scientific literature, for example, the Journal of the American Chemical Society, the Journal of Medicinal Chemistry, or the Journal of Biological Chemistry. Specifically, but without limitation, the following abbreviations may be used in the examples and throughout the specification: mg (milligrams); mL (milliliters); μL (microliters); h (hour or hours); and min (minutes).

### Example 1

### Inhibitory Activity of Compound A13 Against EGFR Mutants

Compound A13 was tested against EGFR mutants: (i) three exon 20 insertions: V7693770insGE, D770_N771insNPG, and A763_Y764insFQEA; and (ii) one point mutation: L861Q; using an HTFR KINEASE-TK kit (Cisbio) according to the manufacturer's protocol. The IC₅₀ values determined are summarized in Table 1.

TABLE 1
Inhibitory Activity against EGFR Mutants

| Mutant | IC₅₀ |
|---|---|
| L861Q | 0.36 nM |
| A763_Y764insFQEA | 0.73 nM |
| V769_D770insGE | 0.62 nM |
| D770_N771insNPG | 0.42 nM |

### Example 2

### Inhibitory Activity of Compound A13 Against EGFR Mutants in Ba/F3 Cells

Compound A13 and osimertinib were tested against EGFR mutants: (i) two-point mutations: G719S and L861Q; and (ii) four exon 20 insertions: A763_Y764insFQEA, V7693770insASV, D770_N771insSVD, and H773_V774insNPH in Ba/F3 cell lines using the CELLTITER-GLO luminescent cell viability assay (Promega) according to the manufacturer's protocol. The IC₅₀ values determined are summarized in Table 2.

### Example 3

### Antitumor Activity of Compound A13 in a Xenograft Mouse Model for Lung Cancer

Compound A13 as a single agent was evaluated for lung cancer treatment in a subcutaneous LU0387 lung cancer PDX model using female Balb/c nude mice. Tumor fragments from stock mice were harvested for inoculation into mice. Each mouse was inoculated subcutaneously in the right rear flank with a PDX model LU0387 tumor fragment (2-3 mm in diameter) for tumor development.

TABLE 2
Inhibitory Activity against EGFR Mutants

IC₅₀

| Mutant | A13 | Osimertinib |
|---|---|---|
| G719S | 2 nM | 32 nM |
| L861Q | 5 nM | 22 nM |
| A763_Y764insFQEA | 4 nM | 15 nM |
| V769_D770insASV | 73 nM | 143 nM |
| D770_N771insSVD | 29 nM | 98 nM |
| H773_V774insNPH | 62 nM | 224 nM |

The randomization started when the mean tumor size reached approximately 517.37 mm³. Twenty-four mice were randomly allocated to 8 study groups, with 3 mice per group. The date of randomization was designated as Day 0.

After tumor inoculation, the mice were checked daily for morbidity and mortality. The mice were checked for any effects of tumor growth and treatments on behavior such as mobility, food and water consumption, body weight gain/loss, eye/hair matting, and any abnormalities. Mortality and observed clinical signs were recorded for each individual mouse in detail.

Tumor volumes (TV) were measured twice per week after randomization using a caliper and the volume was calculated using the formula: V=(L×W×W)/2, where V is tumor volume, L is tumor length (the longest tumor dimension), and W is tumor width (the longest tumor dimension perpendicular to L).

Compound A13 was administered orally as a benzoate salt in a citrate/phosphate buffer at pH 4.0 at 20, 40, and 80 mg/kg once daily for 21 days. The results are summarized in Table 3, where RTV represents relative tumor volume.

Compared to the vehicle control, compound A13 at 40 and 80 mg/kg demonstrated significant anti-tumor activity with TGI (tumor growth inhibition) values of 61% (P=0.016 vs. vehicle control) and 107% (P<0.001 vs. vehicle control), respectively. The tumor volume of mice treated with compound A13 at 80 mg/kg was 36 mm³ on Day 21, which was less than that of 86 mm³ on Day 0. Compound A13 was also well tolerated.

TABLE 3
Effect of Compound A13 on Tumor Volumes

| | TV (mm³) | TV (mm³) | |
|---|---|---|---|
| Compound A13 | on Day 0 | on Day 21 | RTV |
| 0 (Vehicle) | 84 ± 6 | 802 ± 213 | 718 ± 207 |
| 20 mg/kg | 86 ± 6 | 602 ± 67 | 517 ± 61 |
| 40 mg/kg | 89 ± 6 | 370 ± 31 | 281 |
| 80 mg/kg | 86 ± 6 | 36 ± 9 | −40 ± 3 |

Using the same xenograft mouse model, the pharmacokinetic (PK) parameters of compound A13 were determined at 80 mg/kg with a single oral dose. For each mouse, blood samples were collected at 0, 1, 2, 4, 8, 16, and 24 h after dosing. The PK parameters determined are summarized in Table 4.

TABLE 4
Pharmacokentics of Compound A13 in Balb/c Mice

| AUC₀₋ₜ | Cₘₐₓ | t_{1/2} | Vz | CLz |
|---|---|---|---|---|
| (μg · L⁻¹ · h) | (μg · L⁻¹) | (h) | (L · kg⁻¹) | (L · h⁻¹ · kg⁻¹) |
| 3601 ± 273 | 1381 ± 432 | 1.7 ± 0.6 | 52 ± 17 | 21.2 ± 1.8 |

### Example 4

Phase L Evaluation of the Safety, Tolerability, Pharmacokinetics, and Efficacy of Compound A13 in Subjects with Locally Advanced or Metastatic Non-Small Lung Cancer

This is an open label, single arm, nonrandomized, dose escalation/extension study, evaluating the safety, tolerability, and pharmacokinetics (PK), as well as efficacy of N-(2-((2-(dimethylamino)ethyl)(methyl)amino)-4-methoxy-5-((4-(8-methylimidazo[1,2-a]-pyridin-3-yl)pyrimidin-2-yl)amino)phenyl)acrylamide A13 as a benzoate salt in human subjects with locally advanced or metastatic non-small lung cancer (NSCLC). The study is conducted in two phases: Phases Ia and Ib.

Phase Ia is a dose escalation phase, evaluating the safety and tolerability and determining the dose limiting toxicities (DLTs) and maximum tolerated dose (MTD) of orally administered compound A13 in subjects with locally advanced or metastatic NSCLC with EGFR T790M. Dose escalation proceeds according to a 3+3 design. Fifteen to thirty subjects are enrolled in Phase Ia. Each treatment cycle is 28 days (4 weeks).

In Phase Ia, each subject starts with 50 mg per day, escalating gradually to 100, 150, 200, and 250 mg per day. After confirming that the subject does not experience a DLT seven days after taking the starting dose at 50 mg per day, the subject takes the subsequent doses to complete the 28-day treatment cycle. At the same dose level, the next subject cannot start to take compound A13 until the last subject in the same dose level starts taking the second dose. Before escalating to the next dose level, at least three subjects have taken the current dose level at least once and have been evaluated for safety. If one of three subjects experiences a DLT at a dose level, additional three subjects are added to the dose level, thus 6 subjects in total for the dose level. If the three additional subjects do not experience a DLT, then the dose escalation continues. If one or more of the three additional subjects experiences a DLT, the dose escalation is discontinued. If two of the three subjects experience a DLT at a dose level, the dose escalation is discontinued.

Phase Ib is a dose expansion phase, evaluating the efficacy of compound A13 in subjects with a locally advanced or metastatic NSLC with EGFR T790M, an ex20ins, or an uncommon mutation (G719X, S768I, or L861Q). Dose levels in Phase Ib are summarized in Table 5. Seventy-six to one hundred eleven subjects are enrolled. Each treatment cycle is 28 days (4 weeks). Each subject takes a dose per day before a meal.

Eligible subjects for the study are ≥18 years of age with locally advanced NSCLC ineligible for operation or radiotherapy, or metastatic NSCLC. For the dose escalation phase, the eligible subjects are 18-65 years of age; have an ECOG from 0-1; and bear EGFR T790M after treated with a first- or second-generation EGFR-TKI (e.g., erlotinib or gefitinib). For the dose expansion phase, the eligible subjects are 18-75 years of age; have an ECOG from 0-2; and bear EGFR T790M after treated with a first- or second-generation EGFR-TKI (e.g., erlotinib or gefitinib), an EGFR ex20ins (treated or untreated), or an uncommon EGFR mutation (G719X, S768I, or L861Q) (treated or untreated).

Additional inclusion criteria for the eligible subjects include (i) absolute neutrophil count (ANC)≥1500 cells/μL; (ii) platelet count≥90,000/μL; (iii) hemoglobin (HGB)≥90 g/L; (iv) international normalized ratio (INR)≤1.5×upper limit of normal (ULN); (v) serum AST and serum ALT of ≤S2.5×ULN)≤2.5×ULN, and TBIL≤1.5×ULN; (vi) serum creatinine≤1.5×ULN or creatinine clearance of ≥50 mL/min (according to Cockcroft and Gault); and LVEF≤5%

TABLE 5
Dosing Levels in Phase Ib

| Co- | | Dose | No. of |
|---|---|---|---|
| hort | Subject | Level | Subjects |
| 1 | NSCLC subjects bearing EGFR T790M, | 50 mg | 6 |
| | previously treated with an EGFR-TKI | | |
| 2 | NSCLC subjects bearing EGFR T790M, | 100 mg | 10-15 |
| | previously treated with an EGFR-TKI | | |
| 3 | NSCLC subjects bearing EGFR T790M, | 150 mg | 10-15 |
| | previously treated with an EGFR-TKI | | |
| 4 | NSCLC subjects bearing an EGFR ex20ins, | 150 mg | 10-15 |
| | previously treated with an EGFR-TKI | | |
| 5 | NSCLC subjects bearing an EGFR ex20ins, | 200 mg | 10-15 |
| | previously treated with an EGFR-TKI | | |
| 6 | NSCLC subjects bearing an EGFR ex20ins, | 200 mg | 10-15 |
| | previously untreated with an EGFR-TKI | | |
| 7 | NSCLC subjects bearing an uncommon EGFR | 150 mg | 10-15 |
| | mutation (G719X, S768I, or L861Q), | | |
| | previously treated with an EGFR-TKI | | |
| 8 | NSCLC subjects bearing an uncommon EGFR | 200 mg | 10-15 |
| | mutation (G719X, S768I, or L861Q), | | |
| | previously treated with an EGFR-TKI | | |

The safety of compound A13 is evaluated by (i) DLT; (ii) treatment-emergent adverse events (TEABs); (iii) clinical laboratory testing (routine blood tests, blood biochemistry, routine urine tests, and blood coagulation); (iv) physical examinations; (v) vital signs (blood pressures, pulses, respiratory rates, body temperatures, and weights); and (vi) electrocardiogram examinations.

The preliminary efficacy of compound A13 is evaluated by (i) objective response rate (ORR; complete remission (CR)+partial remission (PR)); (ii) disease control rate (DCR; CR+PR+stable disease); (iii) duration of response (DOR); (iv) progression-free survival (PFS); and (vi) overall survival (OS).

The PK parameters of compound A13 are determined, including Cₘₐₓ, Tₘₐₓ . . . , AUC, and t_{1/2}.

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A method of treating, preventing, or ameliorating one or more symptoms of lung cancer bearing an EGFR mutation in a subject, comprising administering to the subject in need thereof a therapeutically effective amount of a compound of Formula (f): 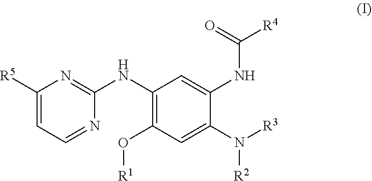
or an enantiomer, a mixture of enantiomers, a diastereomer, a mixture of two or more diastereomers, a tautomer, a mixture of two or more tautomers, or an isotopic variant thereof;
or a pharmaceutically acceptable salt, solvate, hydrate, or prodrug thereof; wherein:
R¹ is C₁₋₆ alkyl or C₃₋₁₀ cycloalkyl;
R² is hydrogen or C₁₋₆ alkyl;
R³ is C₁₋₆ alkyl or heterocyclyl, each independently substituted with amino, C₁₋₆ alkylamino, di(C₁₋₆ alkyl)amino, or heterocyclyl;
R⁴ is C₂₋₆ alkenyl or C₂₋₆ alkynyl; and
R⁵ is bicyclic heteroaryl;
wherein each alkyl, alkenyl, alkynyl, cycloalkyl, heteroaryl, and heterocyclyl is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ is aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₄ alkenyl, C₂₋₄ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

2. The method of claim 1, wherein R⁵ is 5,6- or 6,6-fused heteroaryl, each optionally substituted with one or more substituents Q.

3. The method of claim 1, wherein R⁵ is 5,6-fused heteroaryl, optionally substituted with one or more substituents Q.

4. The method of any one of claims 1 to 3, wherein the compound is a compound of Formula (II): 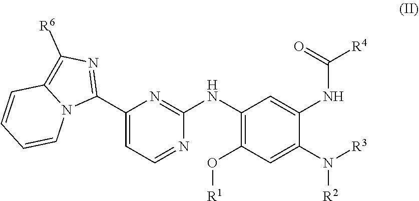
or an enantiomer, a mixture of enantiomers, a diastereomer, a mixture of two or more diastereomers, a tautomer, a mixture of two or more tautomers, or an isotopic variant thereof;
or a pharmaceutically acceptable salt, solvate, hydrate, or prodrug thereof.

5. The method of any one of claims 1 to 3, wherein the compound is a compound of Formula (III): 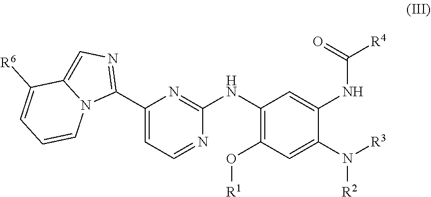
or an enantiomer, a mixture of enantiomers, a diastereomer, a mixture of two or more diastereomers, a tautomer, a mixture of two or more tautomers, or an isotopic variant thereof;
or a pharmaceutically acceptable salt, solvate, hydrate, or prodrug thereof.

6. The method of any one of claims 1 to 3, wherein the compound is a compound of Formula (IV): 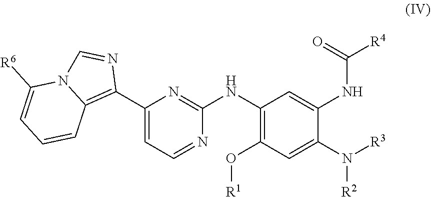
or an enantiomer, a mixture of enantiomers, a diastereomer, a mixture of two or more diastereomers, a tautomer, a mixture of two or more tautomers, or an isotopic variant thereof;
or a pharmaceutically acceptable salt, solvate, hydrate, or prodrug thereof.

7. The method of any one of claims 1 to 3, wherein the compound is a compound of Formula (V): 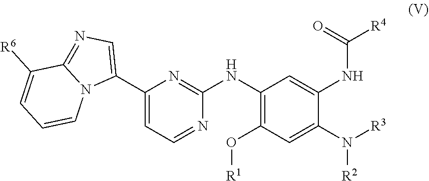
or an enantiomer, a mixture of enantiomers, a diastereomer, a mixture of two or more diastereomers, a tautomer, a mixture of two or more tautomers, or an isotopic variant thereof;
or a pharmaceutically acceptable salt, solvate, hydrate, or prodrug thereof.

8. The method of any one of claims 1 to 3, wherein the compound is a compound of Formula (VI): 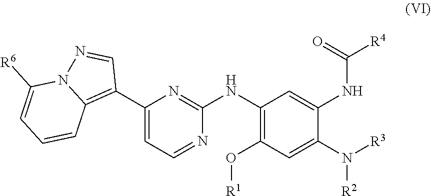
or an enantiomer, a mixture of enantiomers, a diastereomer, a mixture of two or mom diastereomers, a tautomer, a mixture of two or more tautomers, or an isotopic variant thereof:
or a pharmaceutically acceptable salt, solvate, hydrate, or prodrug thereof.

9. The method of any one of claims 1 to 3, wherein the compound is a compound of Formula (II): 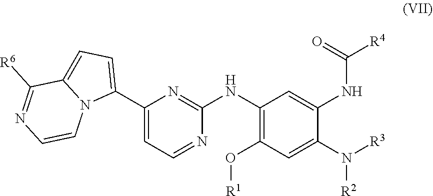
or an enantiomer, a mixture of enantiomers, a diastereomer, a mixture of two or more diastereomers, a tautomer, a mixture of two or more tautomers, or an isotopic variant thereof;
or a pharmaceutically acceptable salt, solvate, hydrate, or prodrug thereof.

10. The method of any one of claims 1 to 9, wherein R¹ is C₁₋₆ alkyl, optionally substituted with one or more substituents Q.

11. The method of any one of claims 1 to 10, wherein R¹ is methyl, fluoromethyl, difluoromethyl, trifluoromethyl, or ethyl.

12. The method of any one of claims 1 to 9, wherein R¹ is C₃₋₁₀ cycloalkyl, optionally substituted with one or more substituents Q.

13. The method of ^{any} one of claims 1 to 9 and 12, wherein R¹ is cyclopropyl.

14. The method of any one of claims 1 to 13, wherein R² is hydrogen.

15. The method of any one of claims 1 to 13, wherein R² is C₁₋₆ alkyl, optionally substituted with one or more substituents Q.

16. The method of any one of claims 1 to 13 and 15, wherein R² is methyl.

17. The method of any one of claims 1 to 16, wherein W is C₁₋₆ alkyl, substituted with amino, C₁₋₆ alkylamino, di(C₁₋₆ alkyl)amino, or heterocyclyl; wherein each alkyl and heterocyclyl is optionally substituted with one or more substituents Q.

18. The method of any one of claims 1 to 17, wherein R³ is 2-dimethylaminoethyl or 2-morpholin-4-ylethyl.

19. The method of any one of claims 1 to 16, wherein R³ is heterocyclyl, substituted with amino, C₁₋₆ alkylamino, or di(C₁₋₆ alkyl)amino; wherein each alkyl and heterocyclyl is optionally substituted with one or more substituents Q.

20. The method of any one of claims 1 to 16 and 19, wherein R³ is 3-dimethylaminnazetidin-1-yl, 3-dimethylaminopyrrolidin-1-yl, 4-dimethylaminopiperidin-1-yl, or 1-methylpiperidin-3-yl.

21. The method of any one of claims 1 to 20, wherein R⁴ is C₂₋₆ alkenyl, optionally substituted with one or more substituents Q.

22. The method of any one of claims 1 to 21, wherein R⁴ is ethenyl or (3-dimethylamino)propen-1-yl.

23. The method of any one of claims 1 to 20, wherein R⁴ is C₂₋₆ alkynyl, optionally substituted with one or more substituents Q.

24. The method of any one of claims 1 to 20 and 23, wherein R⁴ is ethynyl, propyn-1-yl, or (3-dimethylamino)propyn-1-yl.

25. The method of any one of claims 4 to 24, wherein R⁶ is hydrogen or halo.

26. The method of any one of claims 4 to 25, wherein R⁶ is hydrogen or chloro.

27. The method of any one of claims 4 to 24, wherein R⁶ is C₁₋₆ alkyl, optionally substituted with one or more substituents Q.

28. The method of any one of claims 4 to 24 and 27, wherein R⁶ is methyl.

29. The method of any one of claims 4 to 24, wherein R⁶ is C₁₋₆ alkoxy, optionally substituted with one or more substituents Q.

30. The method of any one of claims 4 to 24 and 29, wherein R⁶ is methoxy.

31. The method of any one of claims 1 to 30, wherein the compound is:
N-(2-((2-(dimethylamino)ethyl)(methyl)amino)-4-methoxy-5-((4-(1-methylimidazo[1,5-a]-pyridin-3-yl)pyrimidin-2-yl)amino)phenyl)acrylamide A1;
(E)-4-(dimethylamino)-N-(2-((2-(dimethylamino)ethyl)(methyl)amino)-4-methoxy-5-((4-(1-methylimidazo[1,5-a]pyridin-3-yl)pyrimidin-2-yl)amino)phenyl)but-2-enamide A2;
4-(dimethylamino)-N-(4-methoxy-2-(methyl(2-(methylamino)ethyl)amino)-5-((4-(1-methyl-imidazo[1,5-a]pyridin-3-yl)pyrimidin-2-yl)amino)phenyl)but-2-ynamide A3;
N-(4-methoxy-2-(methyl(2-(methylamino)ethyl)amino)-5-((4-(1-methylimidazo[1,5-a]-pyridin-3-yl)pyrimidin-2-yl)amino)phenyl)but-2-ynamide A4;
N-(2-((2-(dimethylamino)ethyl)(methyl)amino)-4-methoxy-5-((4-(8-methylimidazo[1,5-a]-pyridin-3-yl)pyrimidin-2-yl)amino)phenyl)acrylamide A5;
(E)-4-(dimethylamino)-N-(2-((2-(dimethylamino)ethyl)(methyl)amino)-4-methoxy-5-((4-(8-methylimidazo[1,5-a]pyridin-3-yl)pyrimidin-2-yl)amino)phenyl)but-2-enamide A6;
4-(dimethylamino)-N-(4-methoxy-2-(methyl(2-(methylamino)ethyl)amino)-5-((4-(8-methyl-imidazo[1,5-a]pyridin-3-yl)pyrimidin-2-yl)amino)phenyl)but-2-ynamide A7;
N-(4-methoxy-2-(methyl(2-(methylamino)ethyl)amino)-5-((4-(8-methylimidazo[1,5-a]-pyridin-3-yl)pyrimidin-2-yl)amino)phenyl)but-2-ynamide A8;
N-(2-((2-(dimethylamino)ethyl)(methyl)amino)-4-methoxy-5-((4-(5-methylimidazo[1,5-a]-pyridin-1-yl)pyrimidin-2-yl)amino)phenyl)acrylamide A9;
(E)-4-(dimethylamino)-N-(2-((2-(dimethylamino)ethyl)(methyl)amino)-4-methoxy-5-((4-(5-methylimidazo[1,5-a]pyridin-1-yl)pyrimidin-2-yl)amino)phenyl)but-2-enamide A10;
4-(dimethylamino)-N-(4-methoxy-2-(methyl(2-(methylamino)ethyl)amino)-5-((4-(5-methyl-imidazo[1,5-a]pyridin-1-yl)pyrimidin-2-yl)amino)phenyl)but-2-ynamide A11;
N-(4-methoxy-2-(methyl(2-(methylamino)ethyl)amino)-5-((4-(5-methylimidazo[1,5-a]-pyridin-1-yl)pyrimidin-2-yl)amino)phenyl)but-2-ynamide A12;
N-(2-((2-(dimethylamino)ethyl)(methyl)amino)-4-methoxy-5-((4-(8-methylimidazo[1,2-a]-pyridin-3-yl)pyrimidin-2-yl)amino)phenyl)acrylamide A13;
(E)-4-(dimethylamino)-N-(2-((2-(dimethylamino)ethyl)(methyl)amino)-4-methoxy-5-((4-(8-methylimidazo[1,2-a]pyridin-3-yl)pyrimidin-2-yl)amino)phenyl)but-2-enamide A14;
4-(dimethylamino)-N-(4-methoxy-2-(methyl(2-(methylamino)ethyl)amino)-5-((4-(8-methylimidazo[1,2-a]pyridin-3-yl)pyrimidin-2-yl)amino)phenyl)but-2-ynamide A15;
N-(4-methoxy-2-(methyl(2-(methylamino)ethyl)amino)-5-((4-(8-methylimidazo[1,2-a]-pyridin-3-yl)pyrimidin-2-yl)amino)phenyl)but-2-ynamide A16;
N-(2-((2-(dimethylamino)ethyl)(methyl)amino)-4-methoxy-5-((4-(7-methylpyrazolo[1,5-a]-pyridin-3-yl)pyrimidin-2-yl)amino)phenyl)acrylamide A17;
(E)-4-(dimethylamino)-N-(2-((2-(dimethylamino)ethyl)(methyl)amino)-4-methoxy-5-((4-(7-methylpyrazolo[1,5-a]pyridin-3-yl)pyrimidin-2-yl)amino)phenyl)but-2-enamide A18;
4-(dimethylamino)-N-(4-methoxy-2-(methyl(2-(methylamino)ethyl)amino)-5-((4-(7-methylpyrazolo[1,5-a]pyridin-3-yl)pyrimidin-2-yl)amino)phenyl)but-2-ynamide A19;
N-(4-methoxy-2-(methyl(2-(methylamino)ethyl)amino)-5-((4-(7-methylpyrazolo[1,5-a]-pyridin-3-yl)pyrimidin-2-yl)amino)phenyl)but-2-ynamide A20;
N-(2-((2-(dimethylamino)ethyl)(methyl)amino)-4-methoxy-5-((4-(1-methylpyrrolo[1,2-a]-pyrazin-6-yl)pyrimidin-2-yl)amino)phenyl)acrylamide A21;
(E)-4-(dimethylamino)-N-(2-((2-(dimethylamino)ethyl)(methyl)amino)-4-methoxy-5-((4-(1-methylpyrrolo[1,2-a]pyrazin-6-yl)pyrimidin-2-yl)amino)phenyl)but-2-enamide A22;
4-(dimethylamino)-N-(4-methoxy-2-(methyl(2-(methylamino)ethyl)amino)-5-((4-(1-methyl-pyrrolo[1,2-a]pyrazin-6-yl)pyrimidin-2-yl)amino)phenyl)but-2-ynamide A23;
N-(4-methoxy-2-(methyl(2-(methylamino)ethyl)amino)-5-((4-(1-methylpyrrolo[1,2-a]-pyrazin-6-yl)pyrimidin-2-yl)amino)phenyl)but-2-ynamide A24;
N-(5-((4-(imidazo[1,2-a]pyridin-3-yl)pyrimidin-2-yl)amino)-4-methoxy-2-(methyl(2-morpholinoethyl)amino)phenyl)acrylamide A25;
N-(4-methoxy-2-(methyl(1-methylpiperidin-3-yl)amino)-5-((4-(8-methylimidazo[1,2-a]-pyridin-3-yl)pyrimidin-2-yl)amino)phenyl)acrylamide A26;
N-(5-((4-(8-chloroimidazo[1,2-a]pyridin-3-yl)pyrimidin-2-yl)amino)-2-((2-(dimethylamino)-ethyl)(methyl)amino)-4-methoxyphenyl)acrylamide A27;
N-(2-((2-(dimethylamino)ethyl)(methyl)amino)-4-methoxy-5-((4-(8-methoxyimidazo[1,2-a]-pyridin-3-yl)pyrimidin-2-yl)amino)phenyl)acrylamide A28; or
N-(4-methoxy-2-(methyl(2-(methylamino)ethyl)amino)-5-((4-(8-methylimidazo[1,2-a]-pyridin-3-yl)pyrimidin-2-yl)amino)phenyl)acrylamide A29;
or a tautomer, a mixture of two or more tautomers, or an isotopic variant thereof; or a pharmaceutically acceptable salt, solvate, hydrate, or prodrug thereof

32. The method of any one of claims 1 to 30, wherein the compound is N-(2-((2-(dimethylamino)ethyl)-(methyl)amino)-4-methoxy-5-((4-(8-methylimidazo[1,2-a]pyridin-3-yl)pyrimidin-2-yl)amino)phenyl)acrylamide A13; or a tautomer, a mixture of two or more tautomers, or an isotopic variant thereof; or a pharmaceutically acceptable salt, solvate, hydrate, or prodrug thereof.

33. The method of claim 32, wherein the compound is N-(2-((2-(dimethylamino)ethyl)-(methyl)amino)-4-methoxy-5-((4-(8-methylimidazo[1,2-a]pyridin-3-yl)pyrimidin-2-yl)amino)phenyl)acrylamide A13; or a tautomer, a mixture of two or more tautomers, or an isotopic variant thereof; or a pharmaceutically acceptable solvate or hydrate thereof.

34. The method of claim 33, wherein the compound is crystalline.

35. The method of claim 32, wherein the compound is a pharmaceutically acceptable salt of N-(2-((2-(dimethylamino)ethyl)-(methyl)amino)-4-methoxy-5-((4-(8-methylimidazo[1,2-a]pyridin-3-yl)pyrimidin-2-yl)amino)phenyl)acrylamide A13, or a tautomer, a mixture of two or more tautomers, or an isotopic variant thereof; or a solvate, or hydrate thereof.

36. The method of claim 33, wherein the pharmaceutically acceptable salt is crystalline.

37. The method of any one of claims 1 to 36, wherein the lung cancer is non-small lung cancer.

38. The method of any one of claims 1 to 37, wherein the lung cancer is locally advanced non-small lung cancer.

39. The method of any one of claims 1 to 37, wherein the lung cancer is metastatic non-small lung cancer.

40. The method of any one of claims 1 to 39, wherein the lung cancer is stage II, III, or IV.

41. The method of any one of claims 1 to 40, wherein the lung cancer is unresectable.

42. The method of any one of claims 1 to 41, wherein the lung cancer is refractory.

43. The method of any one of claims 1 to 42, wherein the lung cancer is relapsed.

44. The method of any one of claims 1 to 43, wherein the lung cancer is drug-resistant.

45. The method of any one of claims 1 to 44, wherein the lung cancer is resistant to an EGFR inhibitor.

46. The method of any one of claims 1 to 45, wherein the lung cancer harbors an EGFR mutation in exon 20.

47. The method of any one of claims 1 to 46, wherein the lung cancer harbors an EGFR exon 20 insertion.

48. The method of claim 47, wherein the lung cancer harbors an EGFR exon 20 insertion of A763_Y764insX, and X is an insertion of 1, 2, 3, 4, 5, 6, or 7 amino acids, each independently selected from the twenty natural amino acids.

49. The method of claim 48, wherein the lung cancer harbors an EGFR exon 20 insertion of A763_Y764insFQEA.

50. The method of claim 47, wherein the lung cancer harbors an EGFR exon 20 insertion of V769_D770insX, and X is an insertion of 1, 2, 3, 4, 5, 6, or 7 amino acids, each independently selected from the twenty natural amino acids.

51. The method of claim 50, wherein the lung cancer harbors an EGFR exon 20 insertion of V769_D770insGE or V769_D770insASV.

52. The method of claim 47, wherein the lung cancer harbors an EGFR exon 20 insertion of D770_N771insX, and X is an insertion of 1, 2, 3, 4, 5, 6, or 7 amino acids, each independently selected from the twenty natural amino acids.

53. The method of claim 52, wherein the lung cancer harbors an EGFR exon 20 insertion of D770_N771insNPG or D770_N771insSVD.

54. The method of claim 47, wherein the lung cancer harbors an EGFR exon 20 insertion of H773_V774insX, and X is an insertion of 1, 2, 3, 4, 5, 6, or 7 amino acids, each independently selected from the twenty natural amino acids.

55. The method of claim 54, wherein the lung cancer harbors an EGFR exon 20 insertion of H773_V774insNPH.

56. The method of any one of claims 1 to 55, wherein the subject has not been treated.

57. The method of any one of claims 1 to 55, wherein the subject has been treated.

58. The method of any one of claims 1 to 57, wherein the subject has failed a prior therapy.

59. The method of any one of claims 1 to 58, wherein the subject is a human.

60. The method of any one of claims 1 to 59, wherein the compound is administered orally.

61. The method of any one of claims 1 to 60, wherein the compound is administered as a tablet or capsule.

62. The method of any one of claims 1 to 61, wherein the therapeutically effective amount is ranging from about 0.1 to about 100 mg/kg per day.

63. The method of any one of claims 1 to 62, wherein the therapeutically effective amount is ranging from about 10 to about 1,000 mg per day.

64. The method of any one of claims 1 to 63, wherein the compound is administered in a cycle.

65. The method of any one of claims 1 to 64, wherein one cycle is 28 days.

66. The method of any one of claims 1 to 65, wherein the compound is administered in a 28-day cycle every day for 3 weeks, followed by 1 week of rest.
